(19) United States
(12) Patent Application Publication
(10) Pub. No.: US 2021/0341401 A1
(43) Pub. Date: Nov. 4, 2021

(54) SCINTILLANT NANOPARTICLES FOR DETECTION OF RADIOISOTOPE ACTIVITY

(71) Applicant: ARIZONA BOARD OF REGENTS ON BEHALF OF THE UNIVERSITY OF ARIZONA, Tucson, AZ (US)

(72) Inventors: Craig A. Aspinwall, Tucson, AZ (US); Colleen M. Janczak, Tucson, AZ (US); Zeinab Mokhtari, Tucson, AZ (US); Isen Andrew C. Calderon, Tucson, AZ (US)

(21) Appl. No.: 17/347,938

(22) Filed: Jun. 15, 2021

Related U.S. Application Data
(63) Continuation-in-part of application No. 15/798,183, filed on Oct. 30, 2017, now abandoned.
(60) Provisional application No. 62/477,638, filed on Mar. 28, 2017.
(60) Provisional application No. 62/414,557, filed on Oct. 28, 2016.

(51) Int. Cl. G01N 23/2202; C09K 11/02; C09K 11/06; G01N 33/573

(52) U.S. Cl. CPC G01N 23/2202; C09K 11/02; C09K 11/06; C09K 11/025; G01N 2223/507; C09K 2211/1007; C09K 2211/1018; G01N 2223/07; G01N 33/573

(57) ABSTRACT

Scintillant-doped polystyrene core nanoparticles surrounded by a silica shell can be used to quantify low-energy radionuclides. The nanoparticles are recoverable and re-useable, which may reduce waste and allow for sample recovery. Unlike traditional liquid scintillation cocktail (LSC) formulations, the nanoparticles are made from non-toxic and non-volatile components, and can be used without the aid of surfactants, making them a possible alternative to LSC for reducing the environmental impact of studies that employ radioactive tracers. Recognition elements attached to the functionalized silica surfaces of the nanoparticles allow for separation-free scintillation proximity assay (SPA) applications in aqueous samples. Lipid membrane coatings deposited on the nanoparticle surface can significantly reduce the non-specific adsorption of proteins and other biomolecules, and allow for the incorporation of membrane proteins or other membrane associated binding molecules.

### CROSS REFERENCE

This application is a continuation-in-part and claims benefit of U.S. patent application Ser. No. 15/798,183, filed Oct. 30, 2017, which is a non-provisional and claims benefit of U.S. Provisional Patent Application No. 62/477,638, filed Mar. 28, 2017, and U.S. Provisional Patent Application No. 62/414,557, filed Oct. 28, 2016, the specification(s) of which is/are incorporated herein in their entirety by reference.

### GOVERNMENT SUPPORT

This invention was made with government support under Grant No. R21 EB019133 awarded by NIH. The government has certain rights in the invention.

### FIELD OF THE INVENTION

The present invention relates to surface-modified, core-shell, scintillant-doped nanoparticles for sensitive detection of radioisotope activity, referred to herein as scintillant nanoparticles (SNPs). In one embodiment, the surface modification is a covalent attachment of binding ligands or a specific binding of radiolabeled target molecules or a lipid membrane coating.

### BACKGROUND OF THE INVENTION

Radionuclides, such as ³H, ¹⁴C, ³³P, and ³⁵S, are commonly used as bioanalytical labels and tracers in a wide range of biological, chemical and environmental assays due to the prevalence of H, C, P and S. However, these radioisotopes are challenging to quantify due to their low decay energies (Eₘₐₓ≤300 keV) and short β-particle penetration depths (≤0.6 mm) in aqueous media. Unlike fluorescent or fluorogenic probes and fluorescent protein tags, radionuclides do not significantly increase the size or mass of the labeled component, and therefore have minimal effects on binding, conformational changes, diffusion and active transport, etc. In addition, lower background signals are typically obtained for radioassays compared to fluorescence assays, where background signal arises from the inherent fluorescence of many biomolecules within the sample. Radiolabeling techniques have been used to quantify antigens at sub-pM concentrations, drugs such as buprenorphine at sub-nM concentrations, and enzyme activity.

β-particle emission from radiolabeled analytes is commonly quantified using liquid scintillation analysis (LSA). Scintillation occurs when the energy of a particle (α, β or photon) emitted during radioactive decay is absorbed by absorbing molecules within range, promoting them to an electronically excited state. Relaxation to the ground state results in emission of a photon or the transfer of energy to another molecule, which may then emit a photon. For example, the energy from radioactive decay of radioisotopes is absorbed by aromatic compounds and converted to detectable light, and is ultimately converted to current by a photomultiplier tube (PMT) detector. The energy from radioactive decay is measured in Ci, which is equal to 3.7×10¹⁰ disintegrations per second or 3.7×10¹⁰ Bq.

Typically, aqueous samples are dispersed in scintillation cocktails (LSCs), which are mixtures of aromatic absorbing hydrocarbon molecules (e.g., benzene, toluene, xylene, diisopropylnaphthalene, alkylbezenes), dispersing agents such as surfactants that accommodate the aqueous samples utilized in biological assays, and scintillant fluorophores (molecules that emit photons when excited by energy emitted during radioactive decay, e.g., 2,5-diphenyloxazole or 1,4-Bis(4-methyl-5-phenyl-2-oxazolyl)benzene). As used herein, the term “scintillant fluorophore” may be used interchangeably with “scintillant,” “fluorophore,” or “scintillator.” An approximation of the efficiency of liquid scintillation cocktails is about 10 photons released per 1 keV of emitted beta particles.

One disadvantage of LSC is that it is limited to uses for measurement of ex vivo bulk biological samples due to the cytotoxicity and hydrophobicity of its components. Experiments utilizing LSCs can result in liter or greater volumes of radioactive mixed waste that must be collected and disposed according to state and federal regulations. The toxicity and volatility of the primary solvent components of many LSC formulations complicate their transport, storage, and disposal. When disposing of the waste generated from LSCs, the waste is often classified as mixed waste containing a large quantity of organic solvent, markedly adding to the cost and complexity of disposal. In addition, surfactants can have a negative impact on non-mammalian aquatic organism populations, and may also help spread other pollutants throughout water systems.

Scintillation counting is not limited to samples in LSC, but can also be used for solid scintillators held within a liquid scintillation vial. These solid scintillators can be used instead of liquid scintillation cocktails to reduce toxic effects. Solid scintillators, like the organic scintillant fluorophores described above, absorb energy and emit photons, but are polymers (e.g., polyvinyltoluene or polystyrene) or inorganic crystals (e.g., CdWO₄, CeF₃, yttrium silicate (YSi), or yttrium oxide (YOx)). Inorganic crystalline scintillators have several advantages over polymer-based scintillators, as their high density and high atomic numbers means they can efficiently absorb energy, leading to more sensitive assays. Inorganic scintillants can also absorb higher energy radiation (gamma and X) than polymer-particle based scintillants. However, the density of inorganic crystalline scintillators causes them to settle in storage and sample containers, making it difficult to accurately dispense equal numbers of particles per volume of solution. In addition, samples must be continuously agitated in order to maintain the particles in suspension throughout the time period of the assay.

Although they usually exhibit lower quantum efficiencies than inorganic crystalline scintillators, polymer-based scintillators are less dense and therefore more easily dispensed and dispersed. The molecular structure of polymers limits their use to lower energy radiation such as ³H β-particle emission and Auger electrons emitted by ¹²⁵I, which are considered safer due to their reduced penetration distances in air, water, and tissue. Further, polymer scintillators can incorporate scintillants to which the energy absorbed by the polymer can be transferred, resulting in photon emission at characteristic wavelengths. However, a drawback to the solid scintillant materials is that the majority of the scintillant is unused due to the low penetration depth for many β-particles. The low penetration depth proves particularly problematic for ³H β-particles, which are difficult to detect prior to energy dissipation.

To overcome this limitation, scintillation proximity assays (SPAs) particles are utilized for detection of low energy β-particles from radiolabeled analytes via conjugation of specific binding elements (i.e. radiolabeled analytes) to a particle surface of a solid scintillator, which increases the probability of energy absorption by the scintillant. When the β-particle of the bound analyte is emitted close to the surface (within the penetration distance), the efficiency of detection is markedly increased. SPA is particularly useful for β-particles with low penetration depths (approximately 0.5 μm for β particles emitted from ³H in water) and results in an increased number of emitted photons upon analyte binding. SPA also eliminates the need to separate bound from unbound analytes in radioimmunoassays, markedly enhancing the throughput and simplicity of the assay. Consequently, SPA lends itself to the monitoring of binding kinetics under steady state conditions, as well as to quantification of radiolabeled analytes using automation and high-throughput screening methods.

Modern SPA particle formats utilize scintillant-doped polyvinyltoluene (PVT) or polystyrene (PS) particles, or YSi or YOx particles, to which receptors have been covalently attached. SPAs have been successfully used to measure binding and/or binding kinetics of enkephalins, thyroxin, morphine, inositol phosphates, and many other analytes. However, current SPA platforms are in the micron size regime and thus the majority of scintillant encapsulated within the particle is not utilized due to the sub-micron penetration depths. Further, the lower solubility of polymer microspheres coupled with the more difficult surface modification chemistries complicate utilization and increase the price of the particles. Finally, the large size (5-10 μm diameters) and high density of SPA particles, relative to cells, prevents utilization for intracellular radioisotope detection.

Currently available SPA assays have no mechanism for integration of membrane proteins, many of which are implicated in a variety of diseases and are critical to the development of drug treatments, but are unstable and inactive if removed from a cell membrane-like environment. Lipids have been previously used to coat silica particles and lipid membranes can be stabilized by incorporating polymerizable groups into the lipid structure itself, by crosslinking hydrophobic monomers within the inner leaflet of the membrane, or by combining a polymerizable lipid with a hydrophobic crosslinker. Stabilization of the membrane could reduce coating degradation during storage or use under adverse conditions (centrifugation, surfactant, sonication, drying etc). Hence, there exists a need for scintillant materials that are non-toxic and easily modifiable when used in measuring low-energy radionuclide activity.

### SUMMARY OF THE INVENTION

It is an objective of the present invention to provide nanosensors for dynamic intracellular imaging based on scintillation proximity assays (nanoSPA). The nanoSPA particles and their geometry possess unique and inventive technical features that can overcome several key limitations in radioisotope detection via commercially available LSC, SPA and solid scintillants. These advantages over existing scintillation technologies include, but are not limited to, the following:
1) enhanced compatibility with aqueous samples;
2) the ability to recover from solution following the assay to reduce the waste disposal volume and composition;
3) improved detection efficiency of low-energy β-particle emission using nanoSPA particles having a high surface area to volume ratio;
4) ease of production and modification;
5) an easily modified surface for attachment of biomolecules and other chemical species;
6) a smaller size and high signal output facilitate intracellular detection, which is an unprecedented assay;
7) compatibility with real-time, intracellular radioisotope detection; and
8) use of membrane coatings to reduce non-specific adsorption of protein and other molecules, and to maintain the stability and functionality of membrane proteins/membrane associated binding elements.

Without wishing to limit the invention to a particular theory or mechanism, the highly innovative nanoSPA approach described herein incorporates all key elements for SPA into a small, self-contained nanostructure for real-time studies.

None of the presently known prior references or work has the unique inventive technical features of the present invention. Although SPA beads made of polymers and inorganic crystals are commercially available, they are several μm in diameter (nanoSPA can be <1 μm), which can prohibit intracellular application, and have no mechanism for incorporation of membrane proteins or membrane-specific elements (such as gangliosides like GM1). Other techniques involve the use of solubilized cell membranes, which introduces detergents to the SPA sample.

According to one embodiment, the present invention utilizes polystyrene as the core material due to its chemical compatibility with scintillant fluorophores, which are entrapped therein. The polystyrene matrix facilitates energy absorption and transfer from the radioisotope to the scintillant dye and provides a hydrophobic matrix for maximal scintillation efficiency. A thin (ca. 10-50 nm) silica shell is deposited onto the outside of the polystyrene core of variable diameter (ca. 100-1000 nm). The addition of silica shells to the radioisotope responsive scintillant-doped polymer particles increases solubility in aqueous samples and makes the particles more hydrophilic without using polyhydroxy films or surfactants, which are often required for the dispersion of PVT and PS particles in aqueous samples, thus reducing the aggregation commonly seen with polystyrene nanoparticles. Further still, the silica shell provides an easily modified surface for the covalent attachment of binding ligands or specific binding of radiolabeled target molecules.

According to another embodiment, the present invention features a multifunctional nanosensor platform for dynamic quantification of radioisotope-labeled, membrane protein/membrane associated ligands. The nanoSPA particle may comprise the polystyrene core of variable diameter into which radioisotope-responsive scintillant fluorophores are doped, and on which a silica shell is deposited onto the outside of the polystyrene core. Again, addition of the silica shell increases solubility in aqueous samples, and provides an easily modified surface. The silica shell is also essential for the deposition of a lipid membrane coating on the nanoparticle surface, which not only significantly reduces the non-specific adsorption of proteins and other biomolecules, but is also necessary for the incorporation of membrane proteins or other membrane-associated binding molecules.

Due to their high surface area to volume ratio, the nanoparticles can have greater dynamic ranges and higher efficiency than microwell plate-based designs as well as commercially available scintillant particles, which are often 2 to 10 microns in diameter. Furthermore, the density of the core-shell particles will allow for the particles to remain dispersed in a sample for a longer period of time than scintillating YSi particles, while still providing for easy recovery by centrifugation, thereby simplifying waste disposal and facilitating possible reuse.

According to one embodiment, the present invention features a method for producing scintillant-doped nanoparticles for detecting radioisotope activity by forming polymer nanoparticles and doping said polymer nanoparticles with scintillators. As used herein, the term “swelling-deswelling” refers to said method of first forming the polymer nanoparticles and subsequently doping the nanoparticles with scintillators. In some aspects, for example, the method may comprise: adding monomers to an aqueous solution; polymerizing the monomers to form polymer core nanoparticles in solution; dissolving scintillators in an organic solvent; adding the scintillators in the organic solvent to the polymer core nanoparticle solution; agitating the mixture of the scintillators in the organic solvent and the polymer core nanoparticle solution, thereby doping the polymer core nanoparticles with the scintillators to form scintillant-doped polymer nanoparticles; and removing the organic solvent from the mixture.

According to one embodiment, the present invention features a method for detecting radioisotope activity in a sample. The method may comprise: preparing scintillant-doped polymer core nanoparticles by the swelling-deswelling methods described herein; combining the scintillant-doped polymer core nanoparticles and the sample in a medium, and counting photon emissions. Without wishing to limit the present invention to any theory or mechanism, radioactive decay of the radioisotopes in the sample generate energetic particles that interact with the scintillant-doped nanoparticles, resulting in the emission of photons. In some embodiments, the energetic particles are particles. In other embodiments, medium is an aqueous solution.

One of the unique and inventive technical features of the present invention is the addition of scintillators after polymerization of the polymer core. Without wishing to limit the invention to any theory or mechanism, it is believed that the technical feature of the present invention advantageously provides for better control of the primary to secondary scintillant ratio, protects sensitive scintillant fluorophores from exposure to reactive radicals, and produces less waste compared to other methods. None of the presently known prior references or work has the unique inventive technical feature of the present invention. Furthermore, the inventive technical features of the present invention contributed to a surprising result. For example, the inventors found that the methods described herein was the only way to add in certain scintillators to a polymer matrix. Some scintillants, such as decacyclene, do not get incorporated into the polymer core during polymerization, but the present invention allows for these scintillants to be incorporated into the polymer core.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### Abbreviations

AAPH or AIBA, 2,2′-azobis-2-methyl-propanimidamide dihydrochloride;

APTES or APTS, 3-aminopropyltriethoxysilane;
biotin-NHS, biotin-N-hydroxysuccinimide;

Bq, Becquerel (SI unit of radioactivity);

CE-UV/FS, Capillary electrophoresis-absorbance or fluorescence detector;

Ci, Curie (non-SI unit of radioactivity);

ConA, concanavalin A;

Cys, Cysteine; CySS, Cystine;

DBS, 2,4-dinitrobenzenesulfonyl;

DLS, Dynamic light scattering;

DMPOPOP or dimethyl POPOP, 1,4-Bis(4-methyl-5-phenyl-2-oxazolyl)benzene;

DOPC, 1,2-dioleoyl-sn-glycero-3-phosphocholine

DTDP, 4,4′-dithiodipyridine;

DTNB, 5,5′-dithiobis-(2-nitrobenzoic acid), also known as Ellman's reagent;

DTT, Dithiothreitol;

Eₘₐₓ, Maximum energy of beta particle;

EDAC, N-(3-Dimethylaminopropyl)-N′-ethylcarbodiimide hydrochloride

FRET, Förster Resonance Energy Transfer;

hCys, Homocysteine;

HPLC-EC, High-performance liquid chromatography with electrochemical detector;

HPLC-MS, High-performance liquid chromatography with mass spectrometry detector;

HPLC-UV/FS, High-performance liquid chromatography-absorbance or fluorescence detector;

LSA, Liquid scintillation analysis;

LSC, Liquid scintillation cocktail;

MES, 2-(N-morpholino)ethanesulfonic acid hydrate

MPTS, 3-mercaptopropyltrimethoxysilane;

NEM, N-ethylmaleimide;

NHS, N-hydroxysuccinimide;

NP, Nanoparticle;

NPM, N-(1-pyrenyl)maleimide;

PMT, Photomultiplier tube;

PS, Polystyrene;

PS-APTS, Polystyrene NPs with 3-aminopropyl triethoxysilane coating;

PS-MPTS, Polystyrene NPs with 3-mercaptopropyl trimethoxysilane coating;

pTP, Para-terphenyl;

PVT, Polyvinyltoluene;

RI, Radioisotope;

SAₘₐₓ, Maximum specific activity of radioisotope;

SBD-F, 7-fluorobenzo-2-oxa-1,3-diazole-4-sulfonate;

SBH, Sodium borohydride;

SDS, Sodium dodecylsulfate;

SHE, Standard hydrogen electrode;

SNP, Scintillating nanoparticles;

SPA, Scintillation proximity assay;

SSA, Solid scintillation analysis;

TCEP, Tris(2-carboxyethyl)phosphine;

TEM, Transmission electron microcopy;

TEOS, Tetraethoxysilane;

THF, Tetrahydrofuran.

### BRIEF DESCRIPTION OF THE DRAWINGS

This patent application contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A shows the morphology and function of polystyrene-core silica-shell scintillating nanoparticles (SNPs). The SNPs are prepared by first synthesizing a polystyrene core doped with scintillating dyes (Blue). A thin silica shell (gray) is then deposited on the outside of the polystyrene core. The size of the SNP is readily tuned from 50-500 nm. β-particles emitted from radiolabeled analyte (green circle) penetrate the thin silica shell exciting the dyes in the polystyrene core, resulting in light emission.

FIG. 1B shows the morphology and function of polystyrene-core silica-shell particle-based SPA. β-particles are emitted isotropically from radiolabeled analyte (green circle), although their low penetration depth in aqueous solution minimizes absorption of energy by scintillant-doped particles. β-particle emission remains isotropic after analyte is bound to scintillant-doped particles by surface-attached receptors, but the probability that energy will be absorbed by the particle increases due to proximity.

FIG. 1C shows non-limiting examples of surface-attached receptors.

FIG. 1D shows the morphology and function of polystyrene-core, silica-shell, lipid membrane-coated nanoSPA. When β-emission occurs in solution, the isotropic nature of the decay, coupled with the low penetration depth leads to a marked reduction in signal. Radiolabeled ligand (green circle) binds to a receptor (red ovals) embedded in a stabilizing lipid membrane deposited on the surface of the particle, ensuring a close proximity to the scintillating core upon β-decay, thus increasing light output.

FIGS. 2A-2B shows transmission electron microscopy (TEM) images of pTP and dimethyl POPOP doped polystyrene-core silica-shell particles (FIG. 2A) and pTP and dimethyl POPOP doped polystyrene particles (FIG. 2B).

FIGS. 3A-3B show scintillation response in counts per minute for polystyrene core (with dimethyl POPOP and pTP, blue diamonds), polystyrene (without dimethyl POPOP and pTP, green triangles), and polystyrene-core silica-shell particles (with dimethyl POPOP and pTP doped in the PS core, red squares) samples. The plots show (A) the activity-dependent response of 4 mg particles (FIG. 3A) and the mass-dependent response to 300 nCi ³H acetic acid (FIG. 3B). The error bars represent the standard deviation of three measurements.

FIG. 4 shows a non-limiting scheme of the polystyrene-core silica-shell particle preparation process. Core polystyrene nanoparticles are first synthesized using a surfactant-free polymerization process. Silica shells are added to the cores in a second reaction.

FIGS. 5A-5B show TEM images of polystyrene core nanoparticles (FIG. 5A) and polystyrene-core silica-shell nanoparticles (FIG. 5B).

FIGS. 6A-6B show plots of the scintillation response, in counts per minute, of LSC, nanoSCINT, recovered nanoSCINT, scintillant fluorophore loaded polystyrene core nanoparticles, and water as a control (FIG. 6A). Because the efficiency of LSC is significantly higher than any of the nanoparticle samples, the data for LSC is removed in the plot shown in FIG. 6B so that the response of the particles can be seen.

FIG. 7 is an illustration of the relative geometries of nanoSCINT particles dispersed in an aqueous sample (left) and aqueous sample droplets dispersed in LSC (right). 8-particle emission occurs isotropically, and emitted β-particles are surrounded by the scintillating medium for aqueous sample droplets dispersed in LSC. The chance of energy absorption by nanoSCINT particles dispersed in aqueous sample must be much lower.

FIGS. 8A-8B are plots showing zeta potential measurements of nanoSCINT particles, polystyrene core particles without silica shells, and silica particles without polystyrene cores (FIG. 8A), and scintillation response in counts per minute of nanoSCINT particles at varying pH and high and low salt concentrations (FIG. 8B).

FIGS. 9A-9C show TEM images of PS nPs without shells (FIG. 9A), PS nPs with smooth, amine functionalized shells (FIG. 9B), and PS nPs with rough thiol functionalized shells (FIG. 9C).

FIGS. 10A-10B show nanoSPA for ³H-labeled NeutrAvidin. FIG. 10A is an illustration depicting the binding of ³H-labeled NeutrAvidin to biotin functionalized nanoSPA particles leading to measurable emission of photons at visible wavelengths.

FIG. 10B shows the response of nanoSPA particles after incubation with increasing mole amounts (and consequently, increasing activity) of ³H-labeled NeutrAvidin. The lines serve only as guides to the eye.

FIGS. 11A-11C show nanoSPA for ³H-labeled DNA oligomers. FIG. 11A is an illustration depicting the binding of ³H-labeled DNA oligomer (either the complementary oligomer, or the 4-base pair mismatched oligomer to the oligomer immobilized on the nanoSPA particle surface. FIG. 11B is the response of nanoSPA particles after incubation with increasing mole amounts of ³H-labeled complementary or ³H-labeled 4-base pair mismatched oligomers. FIG. 11C shows the same data of FIG. 11B, but in terms of ³H-labeled oligomer activity, as labeling efficiencies where not equivalent. The lines serve only as guides to the eye.

FIG. 12 shows a scintillation response of uncoated (lipid to particle surface area ratio of “0”) and lipid coated nanoSPA particles after incubation with ³H-labeled BSA (0.8 nmoles at 256 nCi activity). The blue line represents the average background signal for nanoSPA in the absence of ³H.

FIG. 13 shows a scintillation response lipid membrane coated nanoSPA upon binding of cholera toxin B to GM1 inserted in the lipid membrane at 1.0% GM1 (blue triangles) and 0.1% GM1 (red squares), compared to 0% GM1 (green circles). The error bars represent the standard deviations of the measurements of three samples

FIGS. 14A-14C show TEM images of PS (FIG. 14A), PS-MPTS (FIG. 14B), and PS-APTS (FIG. 14C) nanoparticles.

FIG. 15 shows a non-limiting reaction scheme of cysteine binding to PS-MPTS NPs. Blue and red circles inside the PS-MPTS NPs represent pTP and DMPOPOP primary and secondary scintillant fluorophores, respectively.

FIG. 16A shows scintillation counts from 35S-cysteine added to NPs. Red squares: specific binding to PS-MPTS; Blue diamonds: non-proximity effect to PS-APTS; Green circles: response enhancement due to specific binding.

FIG. 16B shows a decrease in scintillation intensity on PS-MPTS NPs (circles) as a result of disulfide cleavage by TCEP (blue) and DTT (green) and disulfide exchange by unlabeled cysteine (red). Scintillation intensity stays constant on PS-APTS (diamonds).

FIG. 16C shows binding of 35S-cysteine to PS-MPTS at different pH values. Error bars represent the standard deviation of three measurements.

FIG. 17 shows a non-limiting reaction scheme of pH-dependent thiol-disulfide exchange.

FIG. 18 shows a non-limiting reaction scheme of binding of thiol-blocked cysteine and thiol-blocked PS-MPTS that is inhibited.

FIG. 19A shows scintillation for samples with and without NEM. Thiol blocking on ³⁵S-Cys, PS-MPTS NPs, or both.

FIG. 19B shows binding inhibition by thiol blocking with varying concentration of NEM.

FIG. 19C shows scintillation for samples without NEM, with NEM, and with NEM and additional SBH. Data are normalized to the sample without NEM. Error bars represent the standard deviation of three measurements.

FIG. 20A shows scintillation for samples with and without HNO. Thiol blocking on both PS-MPTS NPs and 35S-Cys.

FIG. 208 shows scintillation inhibition by thiol blocking with varying concentration of HNO. Error bars represent the standard deviation of three measurements.

FIG. 21 shows scintillation for the oxidation of thiols by metals. More disulfides and less binding are observed with the addition of metals. Error bars represent the standard deviation of three measurements.

FIG. 22 shows a reaction scheme of ³³P-phosphate transfer from ATPγ³³P to SRC kinase substrate catalyzed by SRC kinase. Pink P represents ³³P.

FIG. 23 shows an SRC kinase activity evaluation by LSA. Positive Control sample contains SRC kinase but Negative Control sample does not.

FIG. 24A shows scintillation counts upon mixing NPs with kinase mixtures made by ATP-mix. FIG. 24B shows scintillation counts upon centrifugation to minimize non-proximity effect by removing excess ATPγ³³P (including data from kinase analysis using COLD ATP). Specific binding (to PS-MPTS-NHS) and non-specific adsorption (to PS-MPTS) of ³³P-phosphorylated SRC substrate. Positive and negative control samples in each pair of samples were prepared with and without SRC kinase (±SRC).

FIG. 25A shows scintillation counts upon mixing NPs with kinase mixtures made by ATPγ³³P. FIG. 25B shows scintillation counts upon centrifugation to minimize non-proximity effect by removing excess ATPγ³³P. Specific binding (to PS-MPTS-NHS) and non-specific adsorption (to PS-MPTS) of ³³P-phosphorylated SRC substrate. Positive and negative control samples in each pair of samples were prepared with and without SRC kinase (±SRC).

FIG. 26A shows scintillation counts upon mixing NPs with kinase mixtures made by ATPγ³³P. FIG. 26B shows scintillation counts upon centrifugation to minimize non-proximity effect by removing excess ATPγ³³P. Non-specific adsorption (to PS-TEOS) and inhibition of non-specific adsorption (to PS-TEOS-DOPC) of ³³P-phosphorylated SRC substrate. Positive and negative control samples in each pair of samples were prepared with and without SRC kinase (±SRC).

FIG. 27A shows scintillation counts upon mixing NPs with kinase mixtures made at varying concentrations of ATPγ³³P. FIG. 27B shows scintillation counts upon centrifugation to minimize non-proximity effect by removing excess ATPγ³³P. Specific binding of ³³P-phosphorylated SRC substrate to PS-MPTS-NHS NPs.

### DESCRIPTION OF PREFERRED EMBODIMENTS

The term “nano” when referring to the average particle size or diameter refers, for example, to average particle sizes of from about 1 nm to about 1000 nm, as understood by one ordinarily skilled in the art. Likewise, the term “micron” when referring to the average particle size refers, for example, to average particle sizes of from about 1 μm to about 500 μm.

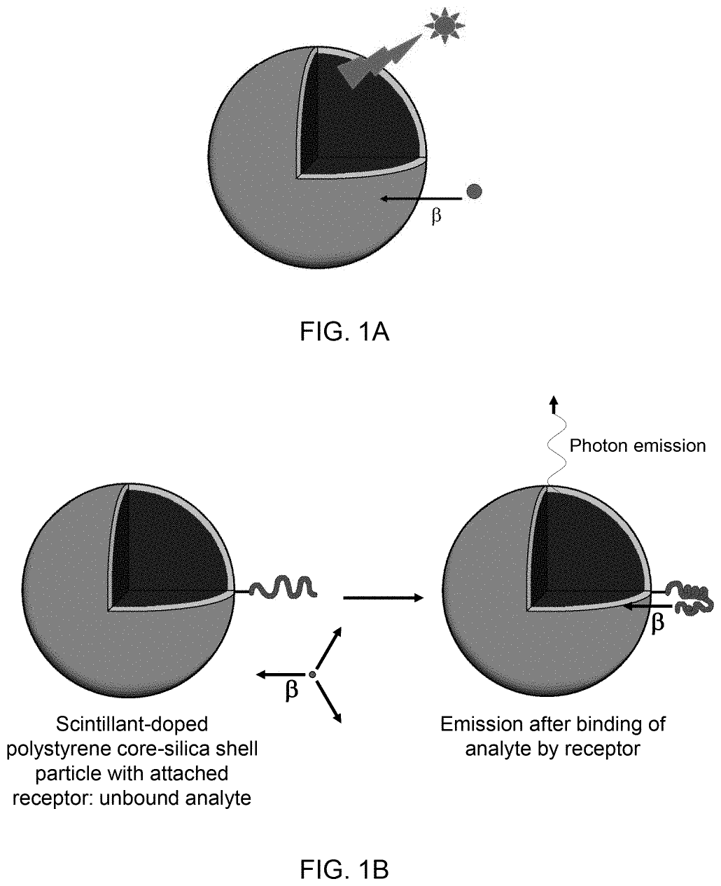

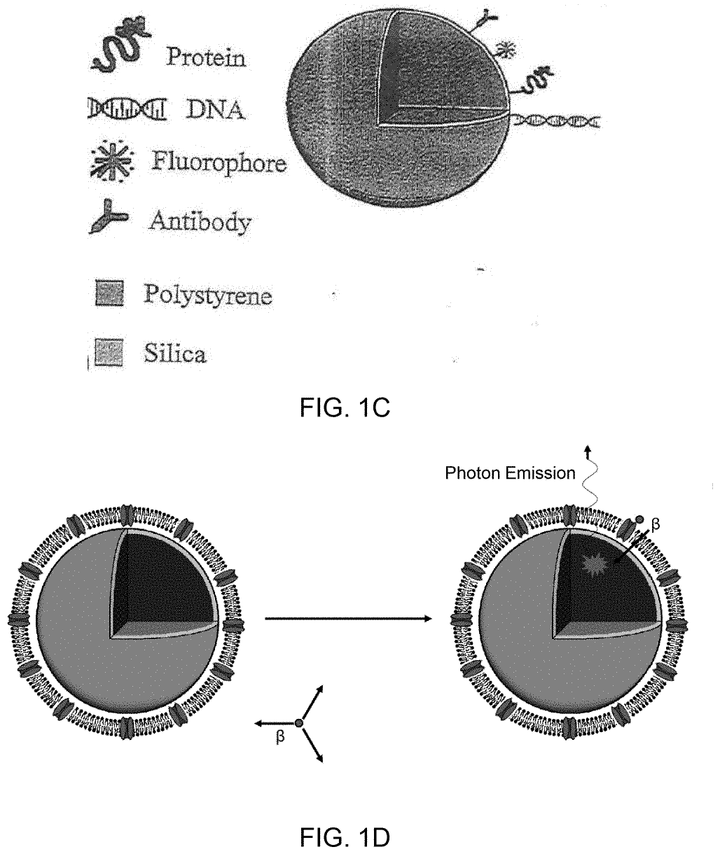

Referring now to FIGS. 1A-27B, in one embodiment, the present invention features a scintillation nanoparticle for detection of radioisotope activity. The nanoparticle may comprise a polymer matrix core, at least one scintillator doped into the polymer core, and a functionalized silica shell encapsulating the polymer core. Without wishing to limit the invention to a particular theory or mechanism, the silica shell is effective for increasing solubility of the scintillation nanoparticle in aqueous solutions. In preferred embodiments, the scintillation nanoparticle may further comprise a surface modifier disposed on a surface of the core-shell particle.

In some embodiments, the polymer matrix core may be comprised of polystyrene or related aromatic polymers. In preferred embodiments, the scintillation nanoparticle is free of surfactants.

In other embodiments, the scintillator may comprise at least two scintillant fluorophores. The scintillant fluorophores may each absorb and/or emit light at different wavelengths. Without wishing to limit the invention to a particular theory or mechanism, the scintillant fluorophores primarily function to shift the emitted light into a wavelength range that is more easily detectable. For instance, the first fluorophore may emit light at a wavelength of about 300 nm. In another embodiment, the second fluorophore emits light at a wavelength of about 450 nm. In other embodiments, the scintillant fluorophores may enable the scintillator to detect different compounds with the same radioisotopes simultaneously. In further embodiments, the scintillator may comprise about 3 to 6 different types of scintillant fluorophores.

In some embodiments, the scintillator may comprise para-terphenyl (pTP), 1,4-Bis(4-methyl-5-phenyl-2-oxazolyl)benzene (DMPOPOP), or a combination thereof. Other examples of scintillators that may be used in accordance with the present invention includes benzene, naphthalene, anthracene, tetracene, and substituted benzenes, naphthalenes, anthracenes, tetracenes, substituted pyrazolines, oxazoles, phenyloxazolyls, or quinolines. However, the scintillators are not limited to the aforementioned examples, and may be any suitable fluorophore.

In preferred embodiments, a surface of the silica shell is modified with functional groups. For example, the surface of the silica shell may be modified with amine or thiol functional groups. In one embodiment, the amine functionality may be obtained from APTS. In another embodiment, the thiol functionality may be obtained from MPTS. However, the surface functionality may be any other suitable or equivalent moiety obtained from a source that would be known to one of ordinary skill in the art.

In some embodiments, the surface modifier may comprise surface-attached receptors bound to the functionalized silica shell. The surface-attached receptors may be covalently or non-covalently bound to the silica shell. For example, surface-attached receptors may be covalently linked to the amine or thiol functional groups. Examples of these surface-attached receptors include, but are not limited to, proteins, nucleic acid aptamers, small molecules or DNA oligomers. Without wishing to limit the invention to a particular theory or mechanism, each particle composition is specific for an individual analyte based on the type of receptor immobilized onto the particle surface. If there is no surface functionalization, then there is no specificity for isotopes.

According to other embodiments, the surface modifier may comprise a lipid membrane substantially covering a surface of the core-shell particle. As used herein, the term substantially can mean covering at least 50 of the core-shell particle surface. For example, the lipid membrane may cover about 50%-75% or 75%-90% or 90% to 100% of the core-shell particle surface.

In one embodiment, the lipid membrane may comprise a lipid bilayer. In another embodiment, the lipid membrane may further comprise receptors embedded in the lipid bilayer. Without wishing to limit the invention to a particular theory or mechanism, each particle composition is specific for an individual analyte based on the type of receptor. If there is no surface functionalization, then there is no specificity for isotopes. Examples of said receptors include, but are not limited to, membrane protein receptors, growth factor receptors, G-protein coupled receptors, ion channels, lipid-derived receptors, glycoprotein receptors, glycolipids, phospholipids, or a combination thereof.

Without wishing to be bound by theory, the lipid membrane may reduce non-specific adsorption of receptors, and maintain stability and functionality of said receptors and other membrane associated binding elements.

In some embodiments, the lipid bilayer may comprise polymerizable lipid monomers and functionalized lipid monomers. The polymerizable lipid monomers may be sorbyl- or dienoyl-containing lipid monomers such as, for example, 1,2-bis(octadeca-2,4-dienoyl)-sn-glycero-3-phosphocholine or 1,2-bis[10-(2′,4′-hexadieoyloxy)decanoyI]-sn-glycero-2-phosphocholine. The functionalized lipid monomers may be amine-functionalized lipid monomers such as amino(polyethylene glycol) (NH₂—PEG).

In other embodiments, the lipid bilayer may comprise non-polymerizable lipid monomers and polymerized, hydrophobic non-lipid monomers. The non-polymerizable lipid monomers may be cell membrane fragments, 1,2-dioleoyl-sn-glycero-3-phosphocholine (DOPC), 1,2-diphytanoyl-sn-glycero-3-phosphocholine monomers, naturally occurring lipids, or synthetic lipids. In one embodiment, each polymerized, hydrophobic non-lipid monomer may comprise a methacrylate, a styrene, or a combination thereof, and a cross-linking agent. In another embodiment, the methacrylate may be an aliphatic methacrylate, or an aromatic methacrylate such as benzyl methacrylate or naphthyl methacrylate. In yet another embodiment, the cross-linking agent may be a dimethacrylate such as, for example, ethylene glycol dimethacrylate.

In still other embodiments, the lipid bilayer may be a non-polymerizable naturally-occurring lipid bilayer, a synthetic lipid bilayer, or a combination thereof.

In some embodiments the diameter of the polymer core can range from about 50-1000 nm. For example, the diameter of the polymer core may be about 100-300 nm or about 300 nm to 500 nm or about 500 nm to 1000 nm. In other embodiments, the silica shell may have a thickness ranging from about 10-500 nm, such as about 10-50 nm or about 50-100 nm or about 100-300 nm or about 300-500 nm.

Another embodiment of the present invention features a method of detecting radioisotope activity in a sample. The method may comprise providing a scintillant material comprising a plurality of scintillation nanoparticles; combining the scintillation material and the sample in a medium such that radioactive decay of the radioisotopes in the sample generate energetic particles that interact with the scintillation material resulting in the emission of photons; and counting the photon emissions generated by the radioactive decay of the radioisotopes in the sample. Without wishing to limit the present invention to a particular theory or mechanism, the scintillant material is effective for detecting different compounds with the same radioisotopes simultaneously. In preferred embodiments, the scintillation nanoparticles may be any scintillation nanoparticle described herein.

In some embodiments, the energetic particles can be β-particles. In other embodiments, the medium is an aqueous solution. In yet other embodiments, the medium may comprise biological cells. Without wishing to limit the present invention to a particular theory or mechanism, the scintillation material may function as a cellular or intracellular imaging probe.

Another embodiment of the present invention features a method for producing scintillant-doped polymer core nanoparticles for detecting radioisotope activity. In some embodiments, the method may comprise polymerizing monomers to form polymer nanoparticles and doping the polymer nanoparticles with one or more scintillators to form scintillant-doped polymer core nanoparticles. Non-limiting examples of the scintillators include benzene, naphthalene, anthracene, tetracene, substituted benzenes, substituted naphthalenes, substituted anthracenes, substituted tetracenes, substituted pyrazolines, substituted oxazoles, substituted phenyloxazolyls, substituted quinolines, or a combination thereof. However, the scintillators are not limited to the aforementioned examples, and may be any suitable fluorophore.

In other embodiments, the method may further comprise mixing silica precursors with the scintillant-doped polymer core nanoparticles to form a functionalized silica shell that encapsulates each scintillant-doped polymer core nanoparticle, thereby forming scintillation nanoparticles. In some embodiments, the method may further comprise depositing a lipid bilayer on an outer surface of the scintillation nanoparticle such that the outer surface is substantially covered by the lipid bilayer. In one embodiment, the method may comprise embedding receptors in the lipid bilayer. Examples of receptors include, but are not limited to, membrane protein receptors, growth factor receptors, G-protein coupled receptors, ion channels, lipid-derived receptors, glycoprotein receptors, glycolipids, phospholipids, or a combination thereof.

According to another embodiment, the present invention features a method for producing scintillant-doped polymer core nanoparticles for detecting radioisotope activity. In one embodiment, the method may comprise adding monomers to an aqueous solution, polymerizing the monomers to form polymer core nanoparticles in solution, dissolving one or more scintillators in an organic solvent, adding the one or more scintillators in the organic solvent to the polymer core nanoparticle solution, agitating the mixture of the one or more scintillators in the organic solvent and the polymer core nanoparticle solution, thereby doping the polymer core nanoparticles with the one or more scintillators to form scintillant-doped polymer core nanoparticles, and removing the organic solvent from the mixture, thereby forming a concentrated solution of scintillant-doped polymer nanoparticles.

In preferred embodiments, the mixture may be agitated by sonication. However, other techniques of agitation as known to one of ordinary skill in the art may be utilized.

In other embodiments, the step of removing the organic solvent from the mixture may comprise evaporating a portion of the organic solvent, agitating (i.e. by sonication) the remaining mixture, and repeating said steps for a number of iterations. The number of iterations may range from about 5 to 15. Without wishing to limit the present invention to a particular theory or mechanism, this method of removing the organic solvent advantageously provides for improved loading by increasing the contact of the scintillators with the polymer core nanoparticles as the organic solvent is gradually removed.

In some embodiments, the method may further comprise redispersing the concentrated solution of scintillant-doped polymer nanoparticles in a second solvent having a base, and mixing silica precursors into the scintillant-doped polymer nanoparticles dispersed in the second solvent. Without wishing to limit the present invention to a particular theory or mechanism, the silica precursors can form a functionalized silica shell that encapsulates each scintillant-doped polymer nanoparticle, thereby forming the scintillation nanoparticles. In one embodiment, the base is effective for tuning the thickness of the silica shell, wherein the base has a pH ranging from 8-12.

In further embodiments, the method comprises depositing a lipid bilayer on an outer surface of the scintillation nanoparticle such that the outer surface is substantially covered by the lipid bilayer. In another embodiment, the method further comprises embedding receptors in the lipid bilayer. Non-limiting examples of the receptors include membrane protein receptors, growth factor receptors, G-protein coupled receptors, ion channels, lipid-derived receptors, glycoprotein receptors, glycolipids, phospholipids, or a combination thereof.

In one embodiment, the monomers may be styrene monomers that polymerize to form polystyrene core nanoparticles. In another embodiment, the monomers may be any aromatic monomers that polymerize to form aromatic polymer core nanoparticles.

In some embodiments, the scintillator may comprise at least two scintillant fluorophores that can each absorb and/or emit light at different wavelengths. Without wishing to limit the invention to a particular theory or mechanism, the scintillant fluorophores can shift the emitted light into a wavelength range that is more easily detectable. In one embodiment, the first fluorophore may emit light at a wavelength of about 300 nm. In another embodiment, the second fluorophore emits light at a wavelength of about 450 nm. Further still, the scintillant fluorophores may enable the scintillator to detect different compounds with the same radioisotopes simultaneously. In further embodiments, the scintillator may comprise about 3 to 6 different types of scintillant fluorophores.

Non-limiting examples of the scintillators include benzene, naphthalene, anthracene, tetracene, substituted benzenes, substituted naphthalenes, substituted anthracenes, substituted tetracenes, substituted pyrazolines, substituted oxazoles, substituted phenyloxazolyls, substituted quinolines, or a combination thereof. For example, the scintillators may comprise 1,4-Bis(4-methyl-5-phenyl-2-oxazolyl)benzene (DMPOPOP), para-terphenyl (pTP), or a combination thereof. However, the scintillators are not limited to the aforementioned examples, and may be any suitable fluorophore.

Examples of solvents that may be used in the present invention include, but are not limited to tetrahydrofuran, isopropanol, chloroform, acetonitrile, ethyl acetate, toluene, acetone, butanol, ethanol, dichloromethane, or a combination thereof.

In one embodiment, the second solvent may comprise a base that has a pH ranging from 8 to 12. A non-limiting example of said base is ammonium hydroxide. Without wishing to limit the present invention to a particular theory or mechanism, the base may be effective for tuning the thickness of the silica shell.

In some embodiments the diameter of the polymer core can range from about 50-1000 nm. For example, the diameter of the polymer core may be about 100-300 nm or about 300 nm to 500 nm or about 500 nm to 1000 nm. In other embodiments, the silica shell may have a thickness ranging from about 10-500 nm, such as about 10-50 nm or about 50-100 nm or about 100-300 nm or about 300-500 nm.

In one embodiment, the silica precursor may comprise tetraethylorthosilicate (TEOS). In another embodiment, the silica precursor may further comprise functionalized silanes. The amount of functionalized silanes may range from about 5% to 15% volume of the silica precursor. For instance, the silica precursor may comprise about 90% vol of TEOS and about 10% volume of a functionalized silane. In some embodiments, the functional silane may be an aminosilane such as APTS, or a thiol-functionalize silane such as MPTS. In preferred embodiments, a surface of the silica shell is modified with functional groups. Without wishing to limit the present invention to a particular theory or mechanism, the functionalized silanes are effective for modifying the outer surface of the silica shell with functional groups. In one embodiment, the amine functionality may be obtained from APTS. In another embodiment, the thiol functionality may be obtained from MPTS. However, the surface modification may be other suitable or equivalent moieties obtained from sources that would be known to one of ordinary skill in the art.

In some embodiments, the method may further comprise depositing a surface modifier on the surface of the functionalized silica shell. In one embodiment, the step of depositing a surface modifier on the surface of the functionalized silica shell may comprise attaching receptors to the outer surface of the functionalized silica shell. The receptors can be covalently or non-covalently bound to the surface. For example, these surface-attached receptors may be covalently linked to the functional groups of the silica shells, such as the amine or thiol functional groups. Without wishing to limit the invention to a particular theory or mechanism, the type of receptor makes the scintillation nanoparticle specific for an individual analyte. Examples of these surface-attached receptors include, but are not limited to, proteins, nucleic acid aptamers, small molecules or DNA oligomers.

In another embodiment, the step of depositing a surface modifier on the surface of the functionalized silica shell may comprise depositing a lipid membrane on an outer surface of the scintillation nanoparticle such that the outer surface is substantially covered by the lipid membrane. For instance, the lipid membrane may be deposited on the outer surface of the scintillation nanoparticle using vesicle fusion techniques. Vesicle fusion techniques are known to one of ordinary skill in the art. In some embodiments, the lipid membrane may cover about 50%-75% or 75%-90% or 90% to 100% of the outer surface.

In some embodiments, the lipid membrane may comprise a lipid bilayer. In other embodiments, the lipid membrane may further comprise receptors embedded in the lipid bilayer. Again, without wishing to limit the invention to a particular theory or mechanism, the type of receptor makes the scintillation nanoparticle specific for an individual analyte. Examples of said receptors include, but are not limited to, membrane protein receptors, growth factor receptors, G-protein coupled receptors, ion channels, lipid-derived receptors, glycoprotein receptors, glycolipids, phospholipids, or a combination thereof. Preferably, the lipid membrane may reduce non-specific adsorption of receptors, and maintain stability and functionality of said receptors and other membrane associated binding elements.

In one embodiment, the lipid bilayer may comprise polymerizable lipid monomers and functionalized lipid monomers. The polymerizable lipid monomers may be sorbyl- or dienoyl-containing lipid monomers such as, for example, 1,2-bis(octadeca-2,4-dienoyl)-sn-glycero-3-phosphocholine or 1,2-bis[10-(2′,4′-hexadieoyloxy)decanoyl]-sn-glycero-2-phosphocholine. The functionalized lipid monomers may be amine-functionalized lipid monomers such as amino(polyethylene glycol) (NH₂-PEG).

In another embodiment, the lipid bilayer may comprise non-polymerizable lipid monomers and polymerized, hydrophobic non-lipid monomers. The non-polymerizable lipid monomers may be cell membrane fragments, 1,2-dioleoyl-sn-glycero-3-phosphocholine (DOPC), 1,2-diphytanoyl-sn-glycero-3-phosphocholine monomers, naturally occurring lipids, or synthetic lipids. In one embodiment, each polymerized, hydrophobic non-lipid monomer may comprise a methacrylate, a styrene, or a combination thereof, and a cross-linking agent. In another embodiment, the methacrylate may be an aliphatic methacrylate, or an aromatic methacrylate such as benzyl methacrylate or naphthyl methacrylate. In yet another embodiment, the cross-linking agent may be a dimethacrylate such as, for example, ethylene glycol dimethacrylate.

In yet another embodiment, the lipid bilayer may be a non-polymerizable naturally-occurring lipid bilayer, a synthetic lipid bilayer, or a combination thereof.

Without wishing to limit the present invention to a particular theory or mechanism, the method of producing the scintillation nanoparticles described herein can advantageously provide for scintillation nanoparticles that are functionalized and have improved dye or fluorophore loading. Further still, the method provides for nanoparticles that are suitably sized to function as cellular or intracellular imaging probes.

### EXAMPLES

The following examples are presented for illustrative purposes only and are not intended to limit the present invention in any way.

### Example 1

The following is a non-limiting example of producing polystyrene core-silica shell nanoparticles using an inclusion method where scintillants were present during formation of the polystyrene core. Equivalents or substitutes are within the scope of the present invention.

Materials

The primary scintillant p-terphenyl (pTP) and secondary scintillant 1,4-bis(4-methyl-5-phenyl-2-oxazolyl)benzene (dimethyl-POPOP) were obtained from Acros Organics (Geel, Belgium). Styrene, biotin-N-hydroxysuccinimide (biotin-NHS), 2,2′-azobis(2-methylpropionamidine) dihydrochloride (AAPH), tetraethylorthosilicate (TEOS), and 3-aminopropyltriethoxysilane (APTS) were obtained from Sigma Aldrich (St. Louis, Mo.). ³H acetic acid (500 mCi/mmol) and ³H glutamic acid (49.6 Ci/mmol) were obtained from MP Biomedicals (Solon, Ohio) and Perkin Elmer (Boston, Mass.) respectively. Sodium dodecylsulfate (SDS) was obtained from Fisher (Pittsburgh, Pa.).

Fabrication of Scintillant-Doped Polystyrene Core nPs

Scintillant-doped polystyrene (PS) core nPs were fabricated via a surfactant-free emulsion polymerization process by combining 6.0 g (58 mmols) of styrene containing 540 mM (3.6 mmols) dimethyl POPOP and 54 mM pTP (360 mmols) for a pTP: dimethyl POPOP ratio of 100:1, 30 mM (0.2 mmols) dimethyl POPOP and 300 mM (2.0 mmols) pTP for a pTP: dimethyl POPOP ratio of 10:1, or 30 mM (2 mmols) dimethyl POPOP and no pTP, with 100 mL of degassed nanopure water in a 500 mL round-bottomed flask equipped with a magnetic stir bar. Polymerization was initiated by adding 6 mg of MPH (220 mM final concentration). The water/styrene combination was stirred briskly under a gentle stream of argon and maintained at 70-80° C. in a water bath for 12 hours. Unpolymerized styrene and some water were removed under reduced pressure. The nPs were rinsed several times with water, isopropanol, and water again. The average diameter of the PS nPs was ca. 175 nm as determined by dynamic light scattering (DLS, Brookhaven Instruments, BI-200 with a BI-DS detector and BI-800AT autocorrelator software, non-negatively constrained least squares, multiple pass calculation). The concentration of nPs in the PS core stock suspension was estimated by freezing and then lyophilizing 4 mL of the suspension, and was found to be approximately 30 mg/mL. PS nPs without scintillants were made according to the same procedure by simply omitting dimethyl POPOP and pTP from the reaction flask.

Addition of Silica Shells to PS Core nPs

Silica shells were added to the PS core nPs by diluting 0.05 mL (1.5 mg) of the PS core stock suspension with 3.95 mL water and 20 mL isopropanol in a 50 mL round bottomed flask. The mixture was stirred briskly using a stir bar and 0.50 mL NH₄OH (28%) was added. After several minutes, 100 mL of a TEOS:APTS combination (90% TEOS, 10% APTS by volume) was added dropwise to the flask. Stirring was continued for 1 hour, after which PS-core silica-shell nPs were collected by centrifugation. The nPs were washed by repeatedly dispersing them in ethanol, centrifuging the ethanol at 14,000 rpm, and removing the supernatant liquid. Ethanol rinsing cycles were followed by three water rinsing cycles. Shells synthesized with TEOS only (no APTS) were fabricated using a similar procedure in which the NH₄OH volume was reduced to 0.25 mL. The average diameter of the PS-core silica-shell nPs was ca. 300 nm as determined by DLS.

Transmission Electron Microscopy

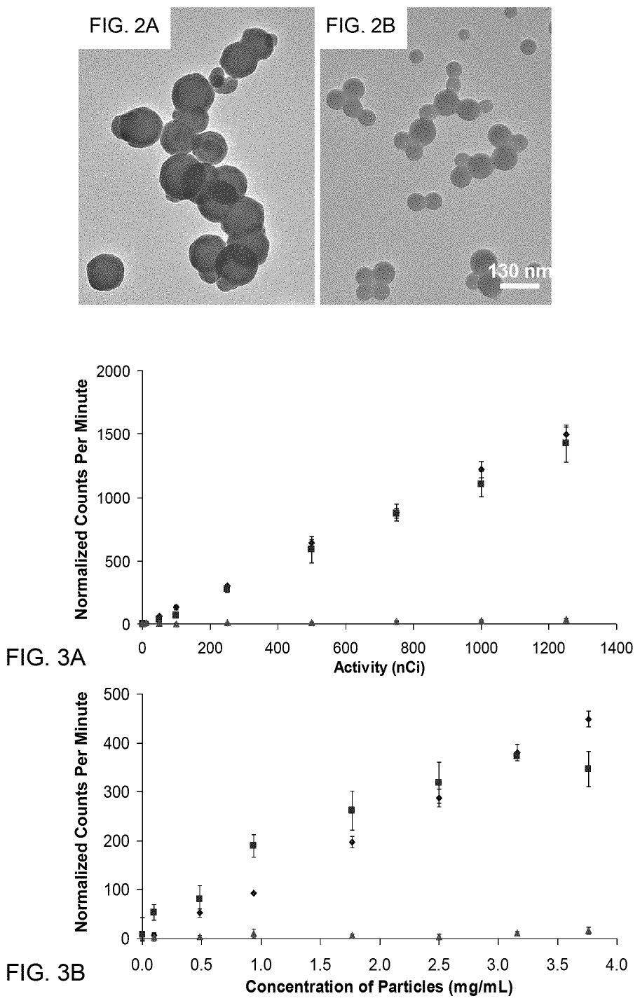

As shown in FIGS. 2A and 2B, PS core and PS-core silica-shell nPs were imaged without staining via TEM (JOEL JEM-100CX-II electron microscope operated at 80 kV accelerating voltage).

Zeta Potential Measurements

Zeta potential measurements were made in disposable folded capillary cells using a Zetasizer Nano (Malvern Instruments Ltd., Worcestershire UK). PS, silica, and PS-core silica-shell nPs were dispersed in 100 mM NaCl at varying pH (from 3 to 10) immediately prior to measurement. Cells were flushed with water and then with 100 mM NaCl at the appropriate pH between samples. Zeta potential was calculated using the Smoluchowski approximation (1.5) as the solution for the Henry equation for all samples.

Comparison of Scintillation Efficiencies for PS Cores with and without pTP

The effect of including pTP along with dimethyl POPOP in PS core nPs was investigated by comparing the scintillation response of PS cores with a ratio of 10:1 pTP (300 mM):dimethyl POPOP (30 mM) to the response of PS cores fabricated with the same concentration of dimethyl POPOP but no pTP. Both the mass-dependent and the activity-dependent responses of the PS core nPs were tested with ³H acetic acid (labeled at the α carbon) using a liquid scintillation counter (Beckman LS 6500, Beckman Coulter, Brea, Calif.). In the mass-dependent response study, 1 mCi of ³H acetic acid was diluted to 10 mL with water. The nP mass was then increased from 0 to 13 mg (0 to 1 mg/mL) by adding volumes of nP stock suspensions to the vials and mixing the solution. The scintillation response in counts per minute was measured for each sample after each addition of nPs. In the activity-dependent study, a fixed mass (10 mg, 1 mg/mL final concentration) of nPs was suspended in 10 mL of water. The activity of the samples was then increased from 0 to 2000 nCi (0 to 190 nCi/mL) by sequential additions of 10 mCi/mL ³H acetic acid. Samples were mixed by aspirating the solution with a pipette and the scintillation response in counts per minute was measured after each addition.

Assay of PS Core and PS-Core Silica-Shell nPs with ³H Acetic Acid

The mass-dependent and activity-dependent scintillation responses of PS core (with dimethyl POPOP and pTP at a ratio of 100:1), PS (without dimethyl POPOP and pTP) and PS-core silica-shell (with dimethyl POPOP and pTP at a ratio of 100:1 in the PS core) were tested with ³H acetic acid. In both studies, all nP samples were dispersed in 1 mL 10 mM SDS solution. The scintillation vials for the Beckman LS 6500 liquid scintillation counter can hold approximately 20 mL, and when only 1 mL sample volumes are used the liquid sample only fills the bottom 1/20 of the vial. Such low volumes in 20 mL scintillation vials may decrease measurement efficiency, as photons emitted from the sample at a the distal edges may not be detected as well as photons emitted from the more central regions of the vial volume. This problem could be alleviated by using larger sample volumes; however, fabricating the amount of nPs needed to complete experiments using larger volumes is impractical. Instead, smaller volume (2 mL) glass autosampler vials were glued to the interiors of 20 mL scintillation vials in such a way as to ensure that 1 mL samples would be contained within a region close to the center of the scintillation vial volume.

In the mass-dependent response study, where mass refers to the mass of PS present rather than the combined mass of both PS and silica, 1 mL aliquots of 10 mM SDS solution were added to modified scintillation vials, along with 33 mL 10 mCi/mL ³H acetic acid (approximately 300 nCi final activity). The nP mass was then increased from 0 to 5 mg (0 to 4 mg/mL) by adding volumes of nP suspensions to the vials and mixing the solution. The scintillation response in counts per minute was measured for each sample after each addition of core-shell nPs.

In the activity-dependent study, a fixed mass (4 mg, based on the mass of PS present) of nPs was added to 1 mL 10 mM SDS solution in modified scintillation vials. The activity of the samples was increased from 0 to 1250 nCi (0 to 890 nCi/mL) by sequential additions of 10 mCi/mL ³H acetic acid. Samples were mixed by aspirating the solution with a pipette and the scintillation response in counts per minute was measured after each addition. Data for both the mass-dependent and activity-dependent studies were normalized to pTP fluorescence intensities at 345 nm (300 nm excitation) in order to account for differences in PS-core silica-shell NP versus PS core NP concentrations.

The response of a fixed mass of scintillant-doped polystyrene-core silica-shell particles to increasing activity of 3H acetic acid (FIG. 3A) and of increasing amounts of scintillant-doped polystyrene-core silica-shell particles to a fixed activity of ³H acetic acid (FIG. 36) is approximately the same as the response of dimethyl POPOP and pTP-doped PS core particles under the same conditions. This similarity indicates that the addition of silica shells to dimethyl POPOP and pTP-doped polystyrene-cores does not prevent or reduce the absorption of energy from δ-particles emitted by ³H acetic acid within the 0 to 1.25 ρCi activity range. It can be seen that polystyrene particles without dimethyl POPOP and pTP do not respond to ³H acetic acid within the same activity range due to lack of scintillant dyes.

Further investigation into the significance of the interactions between the silica surfaces of core-shell NPs and ³H acetic acid was accomplished by comparing the scintillation efficiencies of PS-core silica-shell nPs (10:1 pTP: dimethyl POPOP) made with TEOS and APTS (10% APTS) and PS-core silica-shell NPs made with TEOS only. Equivalent amounts (based on the fluorescence intensity of entrapped pTP excited with 300 nm light) of PS-core TEOS and APTS silica-shell NPs and PS-core TEOS-only silica-shell NPs were diluted to 10 mL with water in unmodified plastic scintillation vials. ³H acetic acid was then incrementally added to each sample to increase the activity from 0 to 70 mCi (0 to 7 mCi/mL). Samples were mixed by aspirating the solution with a pipette and the scintillation response in counts per minute was measured after each addition.

Biotin-Streptavidin Binding Model SPA

Scintillant-doped PS-core silica-shell nPs (ca. 60 mg nPs) were suspended in 5 mL 20 mM pH 8.3 borate buffer with 0.6 mM (3.0 mmols) biotin-NHS for 2.5 hrs. After thorough rinsing and redispersion in water, nPs were treated with 570 nM streptavidin (2.8 nmols). The nP/streptavidin solution was shaken at 120 rpm for 2.5 hrs followed by further rinsing with water. Streptavidin functionalized scintillant-doped PS-core silica-shell nP were then diluted to 33 mL with water. Biotinylated ³H glutamic acid was prepared by diluting 15 mL (15 mCi) 49.6 Ci/mmol ³H glutamic acid with 15 mL 20 mM pH 8.3 borate buffer and 3 mL 20 mM biotin-NHS (60 nmols). ³H glutamic acid and biotin-NHS were allowed to react for 12 hours. A solution containing the same activity of ³H glutamic acid and same concentration of biotin was prepared as a control.

For SPA experiments, 1 mL aliquots of streptavidin-functionalized scintillant-doped PS-core silica-shell nP solution (1.9 mg, or ca. 1.0×10¹¹ nPs each) were placed in modified scintillation vials with 100, 250, 500, 1000, or 3000 nCi biotinylated ³H glutamic acid or ³H glutamic acid and biotin mixture. Water was added to each sample to bring the final sample volume to 1.3 mL. Samples were mixed and allowed to react for 30 minutes before the scintillation response was measured.

### Example 2

The following is a non-limiting example of producing polystyrene-core silica-shell scintillant nanoparticles (nanoSCINT) for low-energy radionuclide quantification in aqueous media. Using a swelling-deswelling process, the scintillants were added after formation of the polystyrene core. Equivalents or substitutes are within the scope of the present invention.

Materials

Styrene, alumina, p-terphenyl (pTP), and 1,4-Bis(4-methyl-5-phenyl-2-oxazolyl)benzene (dimethyl-POPOP) were purchased from Acros Organics.

Tetraethylorthosilicate (TEOS), and 2,2′-azobis(2-methylpropionamidine) dihydrochloride (AIBA), Triton X-100, cycloheaxane, sodium citrate (tribasic) hydrate, sodium tetraborate hydrate, and 2-(N-morpholino)ethanesulfonic acid hydrate (MES) were obtained from Sigma Aldrich. Sodium chloride, sodium phosphate hydrate (monobasic), isopropanol and ammonium hydroxide were obtained from EMD Millipore. Hexanol was purchased from Alfa Aesar. BioCount Liquid scintillation cocktail was acquired from Research Products International. ³H labelled acetic acid (as sodium acetate) was purchased from Perkin Elmer. All chemicals except styrene were used as received. Inhibitor was removed from styrene by passing the styrene through a 3 cm long alumina column immediately prior to use.

Preparation of NanoSCINT Particles

About 3 g of styrene without inhibitor was added to 100 mL degassed water in an argon-flushed 500 mL round-bottomed flask heated to 70° C. in an oil bath. Polymerization was initiated by adding 10 mg of AIBA dissolved in approximately 200 μL of water to the reaction flask. The water/styrene mixture was stirred rapidly throughout the polymerization process, which was allowed to continue for at least 6 hours. Excess styrene and some water were removed from the nanoparticle solution using a rotary evaporator. Polystyrene nanoparticles were doped with scintillant fluorophores by dissolving 53 mg (135 mmoles) of dimethyl POPOP and 262 mg (1.14 mmoles) of pTP in 20 mL of a 10% isopropanol, 90% chloroform solvent mixture. The scintillant fluorophores in solvent were then added directly to the aqueous polystyrene nanoparticle solution in a 500 mL round-bottomed flask. The nanoparticle solution was sonicated in a bath sonicator for several minutes to disperse organic solvent droplets throughout the water. A small amount of the isopropanol and chloroform was then removed using a rotary evaporator. Not all solvent was removed; instead multiple sonication and evaporation cycles were used to allow solvent droplets contact the polystyrene as much as possible and remove the solvent slowly. Once the solvent was removed, the scintillant fluorophore doped polystyrene nanoparticle solution was stored at room temperature until use. A small volume of the solution was removed and lyophilized to determine the weight per volume of nanoparticles.

Silica shells were added to scintillant fluorophore doped polystyrene nanoparticles by dispersing 2 mL of the polystyrene nanoparticle stock solution (approximately 56 mg of nanoparticles) in 200 mL isopropanol with 38 mL water and 5 mL ammonium hydroxide. The dispersion was stirred briskly for several minutes while 1 mL TEOS was added dropwise. Stirring was continued for 1 hour before nanoSCINT particles were collected by centrifugation and rinsed several times with water.

For comparison in zeta potential measurements, silica nanoparticles without polystyrene cores were prepared by dispersing 1.8 mL Triton X-100 in a mixture of 8.0 mL cyclohexane and 1.8 mL hexanol in a 50 mL round bottom flask. The mixture was stirred briskly with a stir bar for several minutes before 550 μL water, 60 μL ammonium hydroxide and 100 μL TEOS were added. Stirring was continued for approximately 12 hours. Silica particles were collected by adding several mL of acetone to cause the particles to flocculate at the bottom of the flask. Particles were rinsed three times with 1.5 mL aliquots of ethanol and then another three times with 1.5 mL aliquots of water. The silica particles were found to be approximately 140 nm diameter by dynamic light scattering.

Transmission Electron Microscopy

Transmission electron micrographs of polystyrene and of nanoSCINT particles were obtained by drying small amount of nanoparticle solutions on carbon films on copper grids. Samples were observed using a Technai G2 Spirit transmission electron microscope (FED.

Zeta Potential Measurements

Zeta potential measurements were made in disposable folded capillary cells using a Zetasizer Nano (Malvern Instruments Ltd., Worcestershire UK). Polystyrene, silica, and nanoSCINT particles were dispersed in 100 mM NaCl at varying pH (from 3 to 10) immediately prior to measurement. Cells were flushed with water and then with 100 mM NaCl at the appropriate pH between samples. Zeta potential was calculated using the Smoluchowski approximation as the solution for the Henry equation for all samples.

Scintillation Efficiency Measurements and NanoSCINT Particle Recovery

The scintillation efficiency of nanoSCINT was tested by combining fixed amounts of nanoSCINT particles in water with ³H-labeled acetic acid. 2 mL of nanoSCINT particle stock solution was added to three 7 mL polyethylene scintillation vials. ³H labeled acetic acid (150 mCi/mmol) was sequentially added to the nanoSCINT samples, as well as to three vials containing only 2 mL each of water and three vials containing 2 mL each of liquid scintillation cocktail. The scintillation efficiency, in counts per minute, was measured for each sample after every addition of ³H acetic acid with a Beckman LS 6000IC liquid scintillation counter. After the final addition of ³H labeled acetic acid, nanoSCINT particles were recovered from each sample by centrifugation. Each recovered nanoSCINT sample was rinsed once by re-suspending the particles in 5 mL fresh water and then collecting them by centrifugation at 16,000 g for 5 minutes. The particles were then suspended again in 2 mL of water and treated with increasing activities of ³H labeled acetic acid as before.

The possible effects of pH and increased salt concentration on nanoSCINT efficiency were explored by dispersing fixed amounts of nanoSCINT particles in 10 mM buffer (sodium citrate at pH 3.0, MES at pH 5.5, sodium phosphate at pH 7.0, and sodium tetraborate at pH 9.5) and measuring scintillation before and after the addition of ³H-acetic acid. Scintillation was measured again after the concentration of NaCl was increased to 100 mM by adding a volume of concentrated NaCl to the same samples.

Liquid scintillation cocktails typically employ the same general process of energy absorption followed by energy transfer and photon emission, although the chemical formulations, which are proprietary, may vary depending on application. The primary component of cocktails is an energy absorbing solvent such as toluene or xylene, or the less flammable and less toxic diisopropylnaphthalene, phenylxylylethane, or dodecylbenzene. Additives such as surfactants are used to facilitate the dispersion of aqueous samples into the organic solvent. Scintillant fluorophores, to which energy absorbed by the solvent is transferred, are included to shift photon emission to wavelengths more readily detected by photomultipliers and CCDs. In the case of solid polymer scintillation particles, the polymer (e.g. polystyrene or polyvinyltoluene) replaces the organic solvent base as the primary absorber of energy from β-particle emission. Although the π-orbital electrons of polystyrene itself are readily excited by β-particle emission, the radiative quantum yield of polystyrene is low (ca. 7%). As with liquid scintillation cocktails, scintillant fluorophores (pTP and dimethyl POPOP) were used to both improve total radiative quantum yield of polystyrene core nanoparticles, and redshift the emission wavelength.

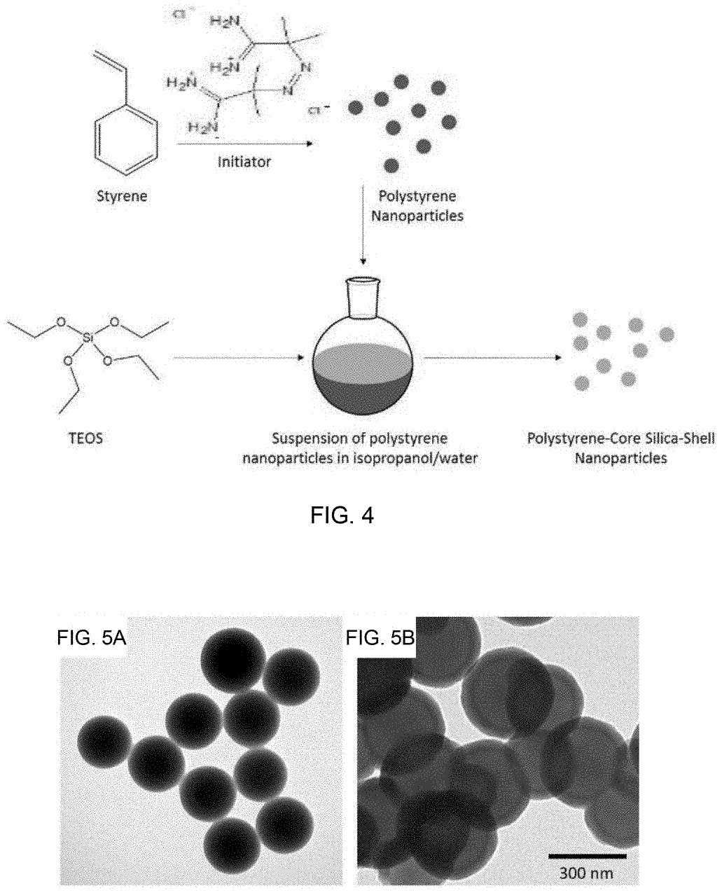

Scintillant-doped polystyrene core nanoparticles were fabricated via surfactant-free emulsion polymerization using the cationic initiator 2,2′-azobis(2-methylpropionamidine) dihydrochloride (AIBA), as illustrated in FIG. 4. The core particles were then doped by swelling the particles with solvent containing dissolved scintillant fluorophores and then slowly removing the solvent, leaving the scintillant fluorophores trapped in the polymer matrix. It has been suggested that surface charge greatly affects the formation of silica shells on polymer particles due to electrostatic interactions between the polymer and the silica oligomers. In fact, initial experiments in which styrene was polymerized using the neutral initiator azobisisobutyronitrile (AIBN) formed mixtures of polystyrene particles and silica nanoparticles during the silica addition step, possibly because neutral polymer composed of styrene polymerized with AIBN has few interactions with the silica precursor molecules or oligomers. In such a case, silica may be more likely to nucleate and grow into nanoparticles in the reaction mixture rather than form silica shells on polystyrene cores. Polystyrene core nanoparticles for nanoSCINT were fabricated using a cationic initiator, which imparts an overall positive charge to the nanoparticles. Cationic particles, such as those composed of styrene polymerized using AIBA, may accumulate anionic silica oligomers and polymeric chains, which develop into silica shells as the hydrolysis and condensation reactions proceed. Silica shells ca. 30 nm thick were deposited on scintillant fluorophore-doped polystyrene core nanoparticles. TEM images of the polystyrene cores before and after the addition of the silica shells can be seen in FIG. 2A and FIG. 2B, respectively.

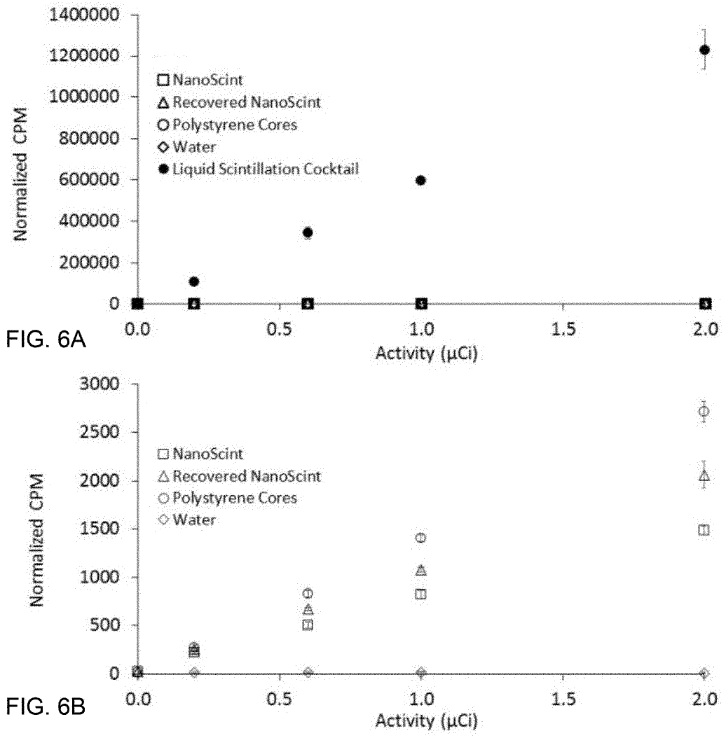

Plots showing the response of nanoSCINT particles and liquid scintillation cocktail in counts per minute as measured by a liquid scintillation counter versus activity can be seen in FIG. 6A-6B. Scintillation efficiency experiments show that while the nanoSCINT particles demonstrate 50 to 100,000 times lower signal than the liquid scintillation cocktail tested (FIG. 6A), they can be used to detect and quantify low-energy β-emitters such as ³H in aqueous solution. The plot shown in FIG. 3B displays the response of scintillant fluorophore-doped polystyrene without the silica shells, nanoSCINT particles, and the same samples of nanoSCINT particles after a single wash with water. Although the response of scintillant fluorophore-doped polystyrene core nanoparticles without silica shells is greater than nanoSCINT, this difference is not attributed to the presence of the silica shell on the nanoSCINT particles. Because sample responses were normalized to the fluorescence of dimethyl POPOP, removal of dimethyl POPOP molecules that may be only adsorbed to the surfaces of the polystyrene nanoparticles after the doping process, or a small amount of leakage of dimethyl POPOP from the polystyrene during the silica shell addition process would reduce the amount of scintillant fluorophore present per number of nanoSCINT particles and lead to the observed difference in scintillation efficiency. The smaller difference between the initial nanoSCINT response and the recovered nanoSCINT response is likely due to differences in the volume of water used to disperse the nanoSCINT particles. Variability in water volume measurement can lead to detectable differences in scintillation efficiency at the sample volume used. A decrease in total volume affects the proximity of nanoSCINT particles to ³H and increases the probability of energy absorption by a nanoSCINT particle during a decay event. Even a difference of 10 μL can, for example, change the distance between a nanoSCINT particle and a ³H labelled molecule by 1.7% (e.g. 130 nm for 1×10¹²nanoSCINT particles in 2 mL), which may be significant when the average traveling distance for a β-particle emitted during ³H decay is only approximately 0.5 μm in water.

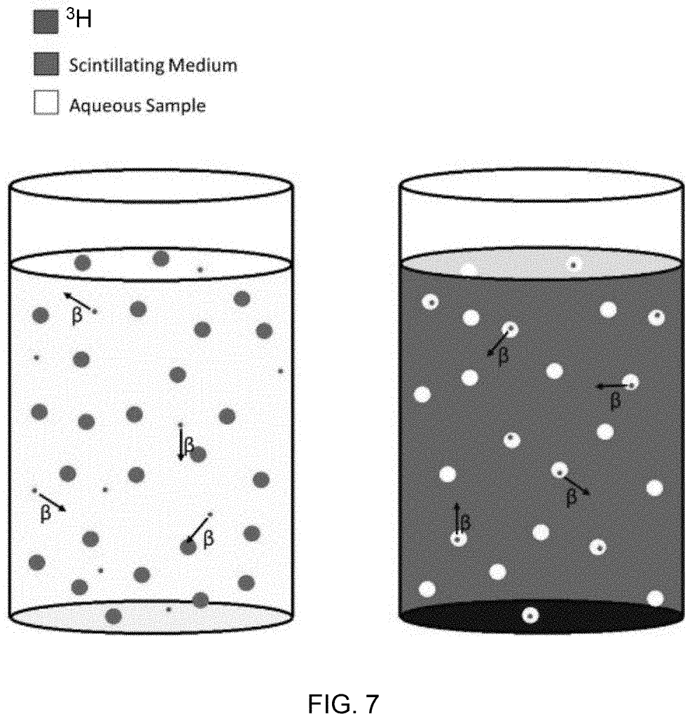

The proximity of individual nanoSCINT particles to ³H labeled molecules affects scintillation response and is also important to consider when comparing nanoSCINT to liquid scintillation cocktail. In 1 mL of a solution containing 4×10¹² nanoSCINT particles, the particles will occupy less than 1% of the sample volume, necessitating the use of either high nanoSCINT concentrations or high radionuclide activities to achieve the same scintillation response as LSC. In contrast, aqueous sample droplets dispersed in LSC will be completely surrounded by the cocktail, which is a more efficient geometry for absorbing energy from isotropic emission. This concept is depicted in the illustration shown in FIG. 7. The compatibility of nanoSCINT particles with aqueous solutions and reusable nature could help reduce the volume of waste that is not only radioactive but also flammable, toxic, and malodorous. Because the bulk of each sample used with nanoSCINT is aqueous, the permeation of solvents from classical liquid scintillation cocktails through plastic vials and waste containers can also be avoided. Furthermore, nanoSCINT particles will not encounter the phase instability/separation problems associated with liquid scintillation cocktails, which have limited capacities for aqueous samples.

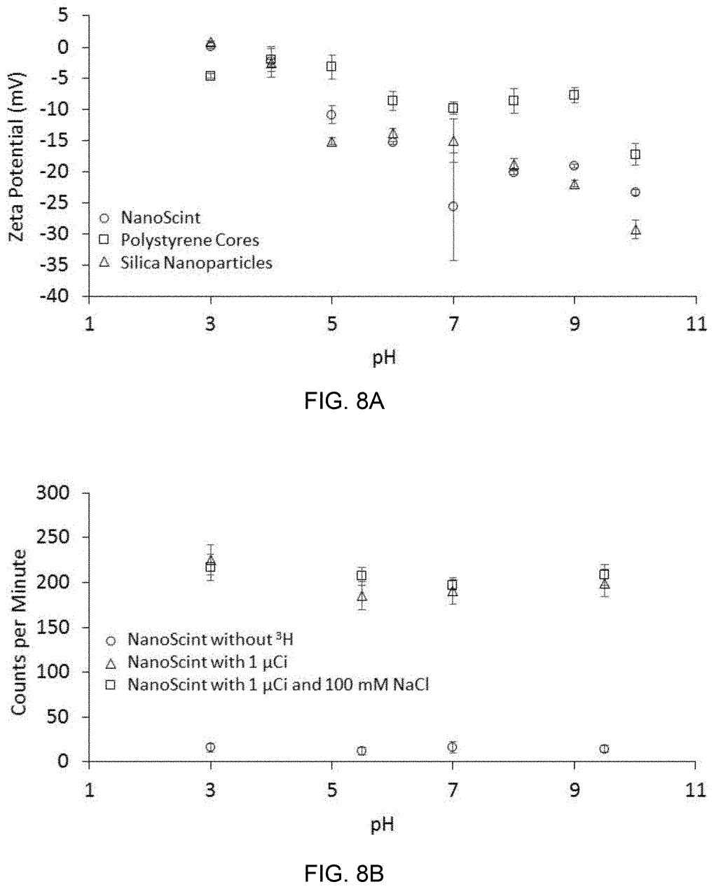

The surface charge characteristics of nanoSCINT were examined by comparing the zeta potentials of nanoSCINT particles to the zeta potentials of silica and polystyrene core nanoparticles (FIG. 8A). The zeta potentials of the silica nanoparticle sample become increasingly negative with increasing pH; at pH 3, the zeta potential is nearly +1.0 mV, while at pH 10 the zeta potential has reached approximately −29 mV. This trend can be attributed to the deprotonation of the silanol groups at the silica nanoparticle surfaces with increasing pH. Interestingly, the zeta potentials of polystyrene core nanoparticles are also negative; the zeta potential at pH 3 is approximately −5.0 mV, and approaches −18 mV at pH 10. As described above, the cationic initiator AIBA was used for the fabrication of polystyrene core nanoparticles in order to make the overall surface charge of the nanoparticles positive. The polystyrene surfaces may therefore be decorated with amidine moieties originating from AIBA that can undergo hydrolysis over time, and with increasing pH, to form amide groups. Consequently, the radius of the electric double layer and the ions contained within that radius may also change. In this case, the electric double layer may become thicker and more diffuse with increasing amidine to amide conversion, resulting in an increase in mobility, and a negative zeta potential of greater magnitude. NanoSCINT particles exhibit a trend very similar to the trends observed for silica nanoparticles and polystyrene core nanoparticles: the zeta potential becomes increasingly negative with increasing pH.

The importance of measuring zeta potential of nanoSCINT particles at varying pH and low and high salt concentrations lies in the stability of an aqueous nanoSCINT dispersion. Although silica is already considered lyophilic, electrostatic repulsions between particles (which are directly related to the fraction of deprotonated silanols and the ions present at the particle surface) at least partially dictate whether particles resist flocculation/aggregation or not. The zeta potential plot shown in FIG. 5A indicates that the surface charge of nanoSCINT particles is close to zero (≤−5 mV) at pH 4 or lower and is only approximately −11 mV at pH 5, which suggests that nanoSCINT particles may aggregate in acidic samples, which in turn may reduce scintillation efficiency by reducing contact with solvated ³H-labeled species. The zeta potential increases with increasing pH as an increasing fraction of the surface silanols become deprotonated, increasing the electrostatic repulsions between particles and possibly stabilizing the dispersion. Although zeta potential measurements were made with nanoSCINT particles dispersed in 100 mM NaCl solution in order to apply the Smoluchowski approximation, the scintillation efficiency of nanoSCINT particles was evaluated for particles dispersed in buffers at different pH, and in the same buffers supplemented with NaCl to a final concentration of 100 mM. Scintillation measurements, the corresponding plots for which are shown in FIG. 5B, show no clear trend with pH for nanoSCINT in buffer only or buffer with 100 mM NaCl. While the total counts per minute do vary from one pH sample to another, with and without 100 mM NaCl, they are not different at the 95% confidence level. These results indicate that the scintillation efficiency of nanoSCINT particles, whether they aggregate or not, is not affected by pH from 3 to 9.5 or by moderate salt concentration, for instance, at least 100 mM NaCl as might be found in many biological samples.

### Example 3

The following is a non-limiting example of a polystyrene-core silica-shell nanoparticle-based SPA platform (nanoSPA). Equivalents or substitutes are within the scope of the present invention.

In a preferred embodiment, a core-shell nanoparticle based scintillation proximity assay platform for the detection of ³H labeled analytes features scintillant fluorophores incorporated into polystyrene core particles surrounded by functionalized silica shells. The functional groups of the silica shells then allow for the covalent attachment of specific binding moieties such as proteins, small molecules, or DNA. The utility of the SPA platform has been demonstrated in two model assays, one in which biotin-functionalized nanoSPA particles are used to measure ³H-labeled Neutravidin, and another in which DNA-oligomer-functionalized nanoSPA particles are used to detect a ³H-labeled complementary strand via hybridization. In both models, nanomole and sub-nanomole amounts of the targets were detected. The nanoSPA platform not only facilitates measurement of ³H-labeled analytes in bulk aqueous solutions, but due to the small diameter of the particles and the protection of the hydrophobic polymer scintillant core by an easily modified silica shell, nanoSPA particles may be used as cellular or intracellular imaging probes.

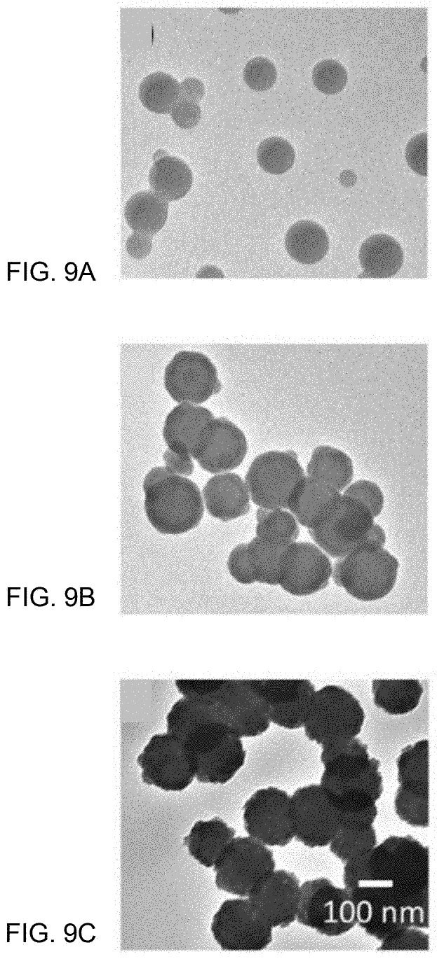

As previously described, the polystyrene cores of nanoSPA particles were prepared in a surfactant-free emulsion polymerization, into which scintillant fluorophores can be incorporated either before polymerization (dissolved in the monomer, styrene) or after polymerization by means of swelling the particles in solvent. pTP was chosen as a primary scintillant fluorophore, and dimethyl POPOP as a wavelength-shifting secondary scintillant fluorophore. Amine or thiol functionalized silica shells were then added to the cores by combining 10% of functional siloxanes (APTS or MPTS) with tetraethylorthosilicate during shell synthesis. As shown in FIG. 9A-9C, TEM images show 150-200 nm scintillant-loaded polystyrene core nanoparticles (PS NPs) surrounded by denser silica shells 25-50 nm thick. While the scintillant-loaded polystyrene cores (FIG. 9A) appear smooth at this magnification, the morphology of the shells can either appear smooth (FIG. 9B) or rough (FIG. 9C) depending on reaction pH and the rates of silica sol nucleation and growth. It was observed that the use of APTS tends to yield smoother shells than MPTS under similar conditions, possibly due to the participation of the amine in the base-catalyzed hydrolysis reaction.

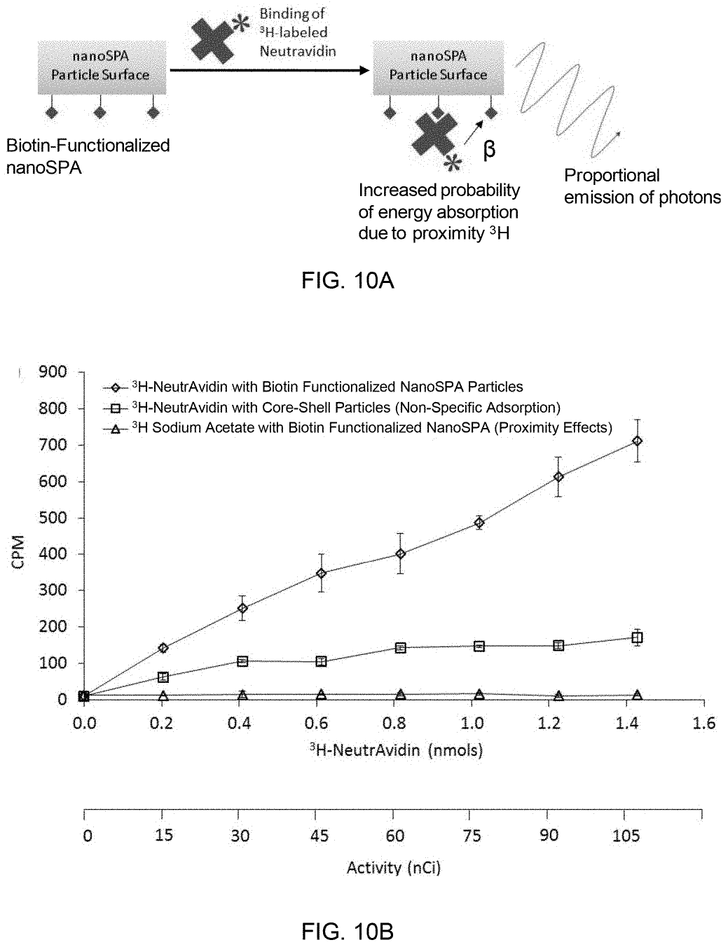

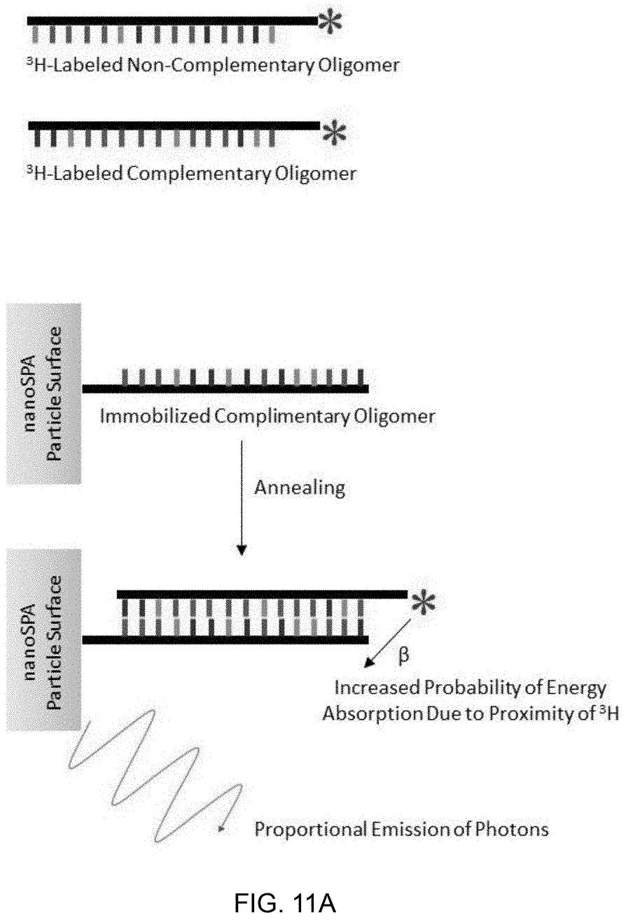

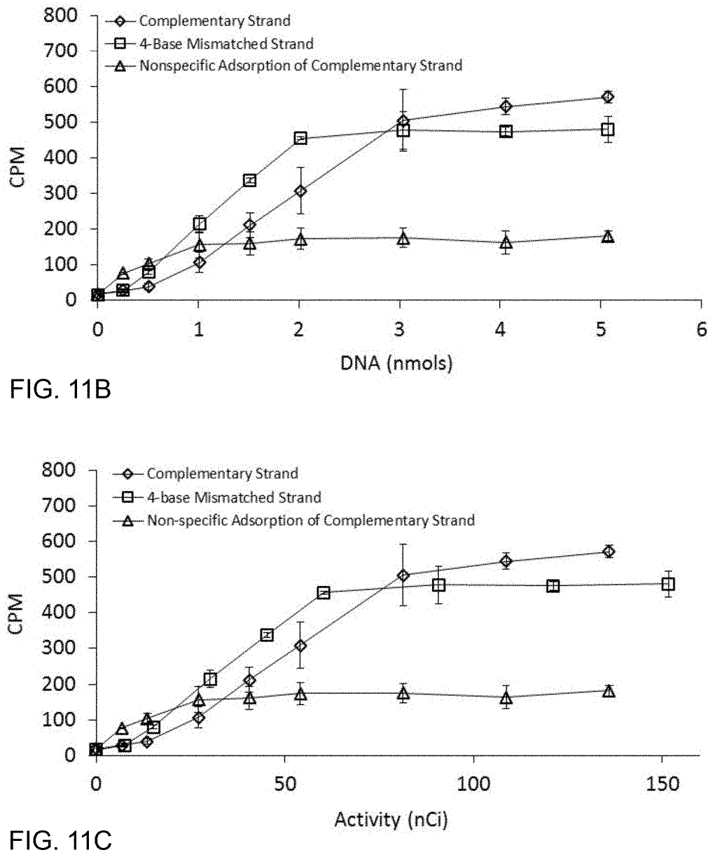

The presence of amine or thiol groups on the silica shells allowed for covalently attaching binding elements to the nanoSPA particle surfaces. Biotin-functionalized nanoSPA particles were used to measure ³H-labeled Neutravidin, and DNA-oligomer-functionalized nanoSPA particles were used to detect a ³H-labeled complementary strand via hybridization. FIGS. 10A-10B and 11A-11B show illustrations of both nanoSPAs, as well as binding curves for biotin-Neutravidin binding and DNA oligomer hybridization. In the biotin/Neutravidin nano SPA model illustrated in FIG. 10A, nanoSPA particles with amine functionalized shells were further modified with biotin N-hydroxysuccinimidyl ester at pH 8. The biotinylated nanoSPA particles were then used to capture ³H-labeled Neutravidin in phosphate buffer. The plot shown in FIG. 10B of counts per minute (CPM) versus nanomoles of Neutravidin and ³H activity shows that not only is scintillation due to non-proximity effects as low as background (generally ≤20 CPM for aqueous solutions), but that scintillation due to non-specific binding of the target is <50% and <25% of the SPA signal at 0.2 and 1.4 nmoles respectively, demonstrating that the increase in CPM with increasing ³H-labeled Neutravidin is in fact due to specific binding of the target to the nanoSPA particles.

The results of the DNA oligomer hybridization nanoSPA model assay are illustrated in FIG. 11A. The plot in FIG. 116 shows significant increases in CPM upon hybridization of the 30-mer immobilized on the nanoSPA particles with increasing amounts of the ³H-labled complementary oligomer, and only approximately 35% of the maximum signal from non-specific adsorption of the ³H-labeled complementary oligomer to particles lacking surface-immobilized oligomer. However, signals due to hybridization of ³H-labeled complementary oligomer and 3H-labeled non-complementary oligomer are very similar: the CPM for ³H-labeled non-complementary oligomer is nearly 95% of that for ³H-labeled complementary strand at the presumed saturation point calculated by assuming that 10% of the particle surface is occupied by functional groups available for oligomer immobilization. The similar SPA results for the complementary and non-complementary oligomers may be attributable to the similarities in sequence, as the non-complementary oligomer has only 4 mismatched bases. Although the penetration depth for ³H β-particles is small, it is likely that any binding, even binding involving only a few base pairs, could lead to an increase in the absorption of energy and a proportional proximity effect that is indistinguishable from complete 30-base pair binding of the complementary oligomer. Possibly, an oligomer with few fewer matched bases would yield a lower SPA signal than the complementary oligomer.

Both the Neutravidin and the complementary DNA oligomer were labeled using the well-studied N-(3-Dimethylaminopropyl)-N′-ethylcarbodiimide hydrochloride (EDAC) and N-hydroxysuccinimide (NHS) coupling reaction, demonstrating that because this method of ³H labeling can be done in-house, nanoSPA could be used even for analytes without ³H labeled versions available for commercial purchase.

NanoSPA particles avoid many of the problems associated with traditional liquid scintillation counting techniques as the scintillator is solid and protected by a hydrophilic silica shell. The easy attachment of recognition elements to the functionalized silica surfaces of the particles makes them versatile for separation-free SPA applications in aqueous samples. In addition, because nanoSPA particles are also small in diameter (<300 nm) and are composed of biologically inert materials, they may potentially serve as sensitive extra- or even intracellular imaging probes.

### Example 4

The following is a non-limiting example of a lipid membrane coated polystyrene-core silica-shell nanoSPA particle. Equivalents or substitutes are within the scope of the present invention.

Materials

Styrene, alumina, p-terphenyl (pTP), and 1,4-Bis(4-methyl-5-phenyl-2-oxazolyl)benzene (dimethyl-POPOP) were purchased from Acros Organics. Tetraethylorthosilicate (TEOS), (3-aminopropyl)triethoxysilane (APIS), (3-mercaptopropyl)triethoxysilane (MPTS), and 2,2′-azobis(2-methylpropionamidine) dihydrochloride (AIBA) were obtained from Sigma Aldrich. Ammonium hydroxide and tetrahydrofuran (THF) were obtained from EMD Millipore. ³H-labeled acetic acid (as sodium acetate) was purchased from Perkin Elmer. Lipids were purchased from Avanti Polar Lipids. All chemicals except styrene were used as received. Inhibitor was removed from styrene by passing the styrene through an alumina column immediately prior to use.

Preparation of Lipid Membrane Coated NanoSPA Particles

3 g of styrene without inhibitor was added to 100 mL degassed water in an argon-flushed 500 mL round-bottomed flask heated to 70° C. in an oil bath. Polymerization was initiated by adding 10 mg of AIBA dissolved in approximately 200 μL of water to the reaction flask. The water/styrene mixture was stirred rapidly throughout the polymerization process, which was allowed to continue for at least 6 hours. Excess styrene and some water were removed from the nanoparticle solution using a rotary evaporator. Polystyrene nanoparticles were doped with scintillant fluorophores by dissolving 53 mg (135 mmoles) of dimethyl POPOP and 262 mg (1.14 mmoles) of pTP in 20 mL of a 10% isopropanol 90% chloroform solvent mixture, or in THF. The scintillant fluorophores in solvent were then added directly to the aqueous polystyrene nanoparticle solution in a 500 mL round-bottomed flask. The nanoparticle solution was sonicated in a bath sonicator for several minutes to disperse organic solvent droplets throughout the water. A small amount of the isopropanol and chloroform was then removed using a rotary evaporator. Not all solvent was removed; instead multiple sonication and evaporation cycles were used to allow solvent droplets contact the polystyrene as much as possible and remove the solvent slowly. Once the solvent was removed, the scintillant fluorophore doped polystyrene nanoparticle solution was stored at room temperature until use. A small volume of the solution was removed and lyophilized to determine the weight per volume of nanoparticles.

Silica shells were added to scintillant fluorophore doped polystyrene nanoparticles by dispersing 2 mL of the polystyrene nanoparticle stock solution (approximately 56 mg of nanoparticles) in 200 mL isopropanol with 38 mL water and 5.0 mL or 7.5 mL (depending on the silica precursors used) ammonium hydroxide. The dispersion was stirred briskly for several minutes while, depending on the desired surface functionality, 2 mL TEOS, TEOS with APTS, or TEOS with MPTS was added dropwise. Stirring was continued for 1 hour before nanoSPA particles were collected by centrifugation and rinsed several times with water.

Lipid membranes were deposited on the silica shells of nanoSPA particles by vesicle fusion techniques. To prepare the vesicles, 3 mg of 1,2-dioleoyl-sn-glycero-3-phosphocholine (DOPC) in chloroform was aliquoted into a vial. The chloroform was removed by evaporation under vacuum overnight. The dried lipid was re-suspended in 1.5 mL nanopure water (18.3 MΩ) to yield a solution with lipid concentration of 2 mg/mL. The lipid solution was sonicated for 10 minutes with 10 minute rest intervals until it became transparent. NanoSPA particles (0.9 mg) were combined with 450 μL of freshly prepared vesicle solution, then sonicated for 5 minutes and stirred overnight to allow lipid vesicles to fuse onto the silica surfaces of the particles. Lipid membrane coated nanoSPA particles were centrifuged (3000 rpm×15 minutes) and re-suspended in buffer (0.1 M sodium phosphate, 0.15 M sodium chloride, pH=7.4) to yield a solution with 0.3 mg/mL particle concentration.

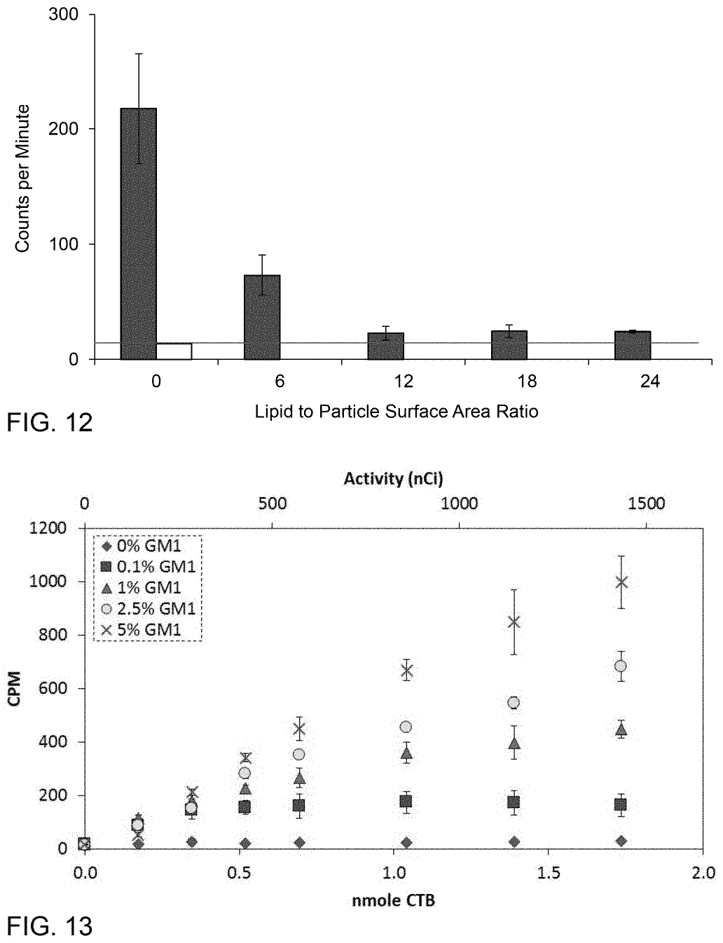

The reduction in non-specific adsorption of protein for lipid membrane coated nanoSPA was evaluated by incubating particles with 3H-labeled bovine serum albumin and then measuring the scintillation response of the particles (FIG. 12). With no lipid membrane present, the response is above 200 counts per minute, a signal background level due to the non-specific adsorption of BSA to the silica surface of the nanoSPA particles. With increasing coverage of the nanoparticle surface by a lipid membrane (controlled by adjusting the lipid to particle surface area ratio), the scintillation response is reduced by approximately 90%, to nearly instrumental background levels. This reduction in non-specific adsorption can significantly improve the detection limit for real ligand-receptor binding events.

The utility of lipid membrane coated nanoSPA has been demonstrated by incorporating the ganglioside GM1 (which has two alkyl chain tails that insert into the leaflet of a lipid bilayer as it is naturally found in animal cell membranes) into the nanoSPA lipid membrane coating for a binding assay with ³H-labeled cholera toxin B, a known GM1 ligand (FIG. 13). The scintillation response for nanoSPA for which 0.1% GM1 has been incorporated into a DOPC lipid membrane coating is approximately 7 times greater than for lipid membrane coated nanoSPA lacking GM1, which is very close to instrumental background. The scintillation response is nearly 16 times greater than GM1-free lipid membrane coated nanoSPA when 1.0% GM1 is incorporated into the membrane coating. Without wishing to limit the invention to a particular theory or mechanism, this low-background, specific binding effect may be observed for other lipid-membrane coated nanoSPA assays with nearly any membrane protein or membrane-associated binding element that can be obtained and inserted into the nanoSPA membrane coating.

### Example 5

The following is a non-limiting example of thiol-responsive scintillation proximity assay core-shell nanoparticles as turn-on biosensors. Equivalents or substitutes are within the scope of the present invention.

Small thiols, such as cysteine (Cys), participate in metabolism, regulation of enzymes, cellular antioxidant defense, signal transduction, protein folding, and coordination of metals. Cys is a regulating factor of thiol-disulfide exchange. It plays a key role in biological activity of S100 proteins and regulates glutathione (GSH) synthesis. Intracellular thiols and disulfides exist primarily in the form of Cys and its dimer cystine (CySS). Cys is the precursor of GSH, concanavalin A(ConA), and taurine. Other biothiols include homocysteine (hCys), small peptides such as cysteinyl-glycine and GSH, and proteins with Cys residues. The free thiol group of Cys makes it one of the most reactive amino acids. Elevated levels of this non-essential amino acid can lead to cardiovascular diseases, neurotoxicity and hypoglycemic brain damage, whereas its deficiency is related to many health problems such as liver damage, muscle and fat loss, and lethargy. Elevated level of Cys is observed in patients with diseases such as Alzheimer's, Parkinson's, cystinuria, and cystinosis and decreased concentration of Cys and CySS is reported in HIV-infected patients. Cys undergoes a reversible redox reaction that can impact protein structure, activity, and interactions with DNA and other proteins. Cys can also reduce metals, such as iron, which can subsequently initiate the redox cycle of iron in the presence of H₂O₂ and lead to DNA damage.

Disulfides are important members of intracellular media and they play key roles in protein folding and function. Disulfide bond formation is mediated by auto-oxidation of thiols, and occurs in cells in specific organelles such as endoplasmic reticulum. Thiol-disulfide exchange is a dynamic process in cells. Thiolate anions, which are the reactive form of thiol, approach disulfide bonds to initiate thiol-disulfide exchange by an S_{N}2 displacement mechanism. The thiol-disulfide ratio is important in maintaining cellular homeostasis and antioxidant defense; otherwise, redox imbalance and oxidative stress will affect the structure and activity of many proteins and cell signaling, which will lead to a variety of diseases such as Alzheimer's, chronic lung disease, atherosclerosis, and age-related macular degeneration.

In order to measure disulfides, thiols that are already present must be protected by blocking agents. N-ethylmaleimide (NEM) is a thiol blocking agent that is commonly used to alkylate thiols through Michael addition, as known to one of ordinary skill in the art. Then, a reducing reagent, such as sodium borohydride (SBH), can be used to cleave the disulfides to thiols for measurement. SBH is a reducing agent that can be used to convert the disulfides to thiols without affecting the NEM-blocked thiols. It also deactivates any excess NEM to prevent the further alkylation of thiols that are newly reduced from the disulfides.

Current optical methods for thiol measurement (HPLC-UV/FS, CE-UV/FS, etc.) are based on derivatization and separation. Fluorimetry detection has been widely used as a sensitive and versatile probe for biothiols. UV-VIS spectrophotometry-based sensitive and selective probes of derivatized biothiols have also been reported. Thiol-specific labels (e.g. 4,4′-dithiodipyridine (DTDP), 7-fluorobenzo-2-oxa-1,3-diazole-4-sulfonate (SBD-F), N-(1-pyrenyl)maleimide (NPM)), 2,4-dinitrobenzenesulfonyl (DBS), and Ellman's reagent (5,5′-dithiobis-(2-nitrobenzoic acid) or DTNB)) are commonly used to label thiols. However, the derivatized thiol might be unstable and light sensitive. Most of these methods that take advantage of nucleophilic interaction of thiol groups with electrophile centers in the derivatizing agent are not selective and the separation step after derivatization is a big drawback. Generally, the derivatizing agents are costly and they require relatively expensive instrumentation for detection. More importantly they can only be employed for in vitro analyses.

Electrochemical measurement and HPLC-MS are other sensitive and selective physical techniques used for the analysis of Cys and other biothiols and do not require derivatization. However, the detector in HPLC-EC is hard to stabilize and MS is an expensive and destructive technique. Enzyme-based techniques are only possible for homocysteine (hCys).

As previously stated, radionuclides are widely used in bioanalytical experiments to tag small molecules lacking optical/electrochemical activity. The mass difference introduced by radiolabels to the target analyte is negligible, as compared to other labeling methods such as fluorescent labeling. Thus, radiolabeling minimizes the perturbation on analyte properties, such as diffusion coefficient, binding kinetics, etc. Beta particles are emitted from ³H, ³⁵S, ¹⁴C, and ³²P, where the maximum energy (Eₘₐₓ) of emitted beta particles is 18 keV for ³H decay, 156 keV for ¹⁴C decay, 167 keV for ³⁵S decay, and 1710 keV for ³²P decay. Radiolabeled biomolecules have very similar chemical, physical, and biological properties to the unlabeled molecules. ³⁵S-Cys, for example, undergoes thiol-disulfide exchange reactions similar to unlabeled Cys with a slightly different rate because of the isotope effect.

Because of the high prevalence of hydrogen in biomolecules, using ³H-labeled analytes is very advantageous. Beta particles emitted from ³H have lower penetration depth (maximum ca. 6 μm in water and 0.5 cm in air), making it a relatively safe isotope. However, it is more challenging to detect this radioisotope due to its low energy beta particles. The ³⁵S isotope, which is a medium-energy isotope, has higher decay energy and therefore its beta particles travel a longer distance (maximum ca. 300 μm in water and 25 cm in air), making ³⁵S a better tracer for sensitive detection of sulfur-containing analytes. Although ³⁵S emits higher energy beta particles, the penetration depth is short enough that this radioisotope is also considered to be safe to work with, and it is commonly used in biological systems. As compared to ³H, ³⁵S is also more convenient to work with because its shorter half-life (87.4 days) allows for easier disposal. LSA is a very efficient technique for measurement of ³⁵S. However, LSCs is not practical or feasible for the same reasons as previously discussed.

Due to the limitations of LSCs, solid scintillation analysis (SSA) may be a better choice for intracellular analysis of the radionuclides with low energy and short range emission. SSA facilitates intimate contact of the radionuclides with the scintillants which may be separated from the analytes for reuse. SPA is used for binding measurements and works based on the conversion of energy released from radionuclides bound to the sensor to detectable visible light. This conversion of energy is done using reporter organic fluorophores and inorganic crystals that transfer the energy via Förster Resonance Energy Transfer (FRET). The reporter fluorophores can be doped into solid matrices such as microplates or micro/nanoparticles. The binding moiety in SPA makes this procedure more sensitive, as compared to SSA.

A sensor with high cell-permeability and low possible interference that can measure intracellular thiols and disulfides and their ratio is desirable. The ideal sensor should have quick and dynamic binding capability so that thiol-disulfide homeostasis is not perturbed over the measurement time.

Material and Instruments

Cystine, L [³⁵S] dissolved in 0.01 N HCl (contains both monomer and dimer) was received from American Radiolabeled Chemicals (St. Louis, Mo.). Ultrapure L-Cys was obtained from Fluka (Milwaukee, Wis.). TCEP, NEM, DTT, TEOS (98%), APTS (99%), MPTS (97%), MPH (97%) and sodium hydrogen phosphate (99%) were purchased from Sigma Aldrich (St. Louis, Mo.). Styrene (99%), pTP (99%), and DMPOPOP (98%) were purchased from ACROS Organics (Geel, Belgium). SBH was a product of Mallinckrodt (St. Louis, Mo.). ACS-grade isopropyl alcohol was purchased from Merck (Kenilworth, N.J.). Biocount LSC was obtained from Research Product International (Mt. Prospect, Ill.). ACS-grade ammonium hydroxide was purchased from EMD Millipore (Billerica, Mass.). Sodium hydroxide (98.9%) was purchased from Avantor Performance Materials (Center Valley, Pa.). Angeli's salt (Na₂N₂O₃) was synthesized as described in Smith & Hein (“The alleged role of nitroxyl in certain reactions of aldehydes and alkyl halides”. Journal of the American Chemical Society 1960, 82 (21), 5731-5740.) Nanopure milliQ water (18 MΩ) was obtained using Easy Pure UV/UF Bamstead.A Tecnai G2 Spirit 20-120 kV transmission electron microscope (TEM) was used to characterize the NPs. A Beckman Coulter LS 6000IC was used for scintillation measurements.

Nanosensor Fabrication and Characterizations

Polystyrene/silica core-shell NPs doped with red-shifted scintillants were fabricated to convert the emitted low energy beta particles from the labeled biomolecule to a visible light via FRET. First, inhibitors were removed from styrene by passing 3 grof liquid styrene through an alumina column. Styrene was then polymerized using a surfactant-free emulsion polymerization method in the presence of scintillant fluorophores (pTP and DMPOPOP with mole ratio of 10:1) in order to produce reporter fluorophore-doped NPs. A cationic initiator, MPH, was utilized to trigger the polymerization of styrene in 50 mL degassed nanopure water overnight. Then any excess unpolymerized styrene was removed from the flask by rotary evaporation. The concentration of PS NPs was obtained by freeze-drying. PS NPs were covered with a silica shell which protects the assembly and increases its hydrophilicity. The silica shell is modifiable so that the surface of the NPs can be decorated with a variety of functional groups that covalently bind to the analyte of interest. Amine and thiol functionalized NPs were prepared using 10% APTS and MPTS, respectively, in addition to 90% TEOS. About 60 mg PS NPs were dispersed in a mixture of 200 mL isopropanol and 40 mL water. 7 or 5 mL NH₄OH was added to adjust the pH for MPTS or APTS, respectively. Then 2 mL of a mixture of 10% MPTS or APTS and 90% TEOS were added dropwise to the PS NPs to obtain PS-MPTS or PS-APTS, respectively. Transmission electron microscopy with accelerating voltage of 100 kV was used to explore the NPs' size and surface morphology on the polystyrene core, and the polystyrene-silica core-shell NPs. TEM samples were prepared on cupper grids of 300-mesh coated with carbon. A drop of diluted suspension was applied to the surface of the grid and left for 10 minutes. Then the residual sample was removed using a piece of fitter paper.

Binding Experiment

Three trials were prepared for all samples in all of the following experiments.

Phosphate buffer (100 mM) pH 7 was prepared and degassed with argon for 20 minutes. ³⁵S-CySS dissolved in 0.01 N HCl that was frozen was thawed and 40 μL of the stock was diluted in 1060 μL buffer. The volumes of reagents were chosen such that the pH stayed buffered after dilution. Solutions of 0, 25, 50, and 100 μL ³⁵S-Cys was added to 1 mL PS-MPTS NPs with concentration of 1 mg/mL. ³⁵S-Cys concentration in these samples ranges from 0 to about 4 nM. A set of control samples with PS-APTS NPs was also prepared to investigate the binding of ³⁵S-Cys to NPs with amine functional groups. All reagents were freshly made and used except for the NPs that are stable over a long time.

Disulfide Cleavage/Exchange

Excess TCEP and DTT (a few mM) were added to the mixture of ³⁵S-Cys and PS-MPTS NPs to cleave the formed bonds. Excess unlabeled Cys (a few mM) was added to the mixture of ³⁵S-Cys and PS-MPTS NPs for disulfide exchange. Data collection was repeated over time to follow the cleavage/exchange reaction.

pH-Dependent Binding

Binding of ³⁵S-Cys to PS-MPTS NPs in three different buffers was performed. For each pH NPs were dispersed in the corresponding buffer and ³⁵S-Cys was diluted in the same buffer. MES (pH 5), PBS (pH 7), and carbonate (pH 9) were chosen for this experiment. About 22 μL of ³⁵S-Cys was diluted in 2 mL of buffer and 650 μL of this solution was added to 1.5 mL of PS-MPTS NPs dispersed in buffer (conc.=1 mg/mL). SPA was measured at different pH values.

Thiol-Blocking by NEM

³⁵S-Cys (˜5 nM) in phosphate buffer pH 7 and PS-MPTS NPs (1 mg/mL) dispersed in the same buffer were separately mixed with excess NEM (˜16 mM) in order to block the thiols. Then the thiol-blocked reagents (³⁵S-Cys and PS-MPTS NPs) were mixed to follow the scintillation inhibition due to binding inhibition. Thiol blocking on both reagents (³⁵S-Cys and PS-MPTS NPs) as well as only one of them were tested. In the NEM concentration-dependent experiment, only the thiols on ³⁵S-Cys (˜1 nM) were blocked. A control sample was also prepared without NEM to observe the scintillation due to the specific binding of ³⁵S-Cys to PS-MPTS. NEM treatment for thiol blocking was performed for 10 minutes and then the thiol-blocked ³⁵S-Cys and PS-MPTS were mixed. The control sample did not have any NEM and ³⁵S-Cys was added to PS-MPTS at the same time as the NEM-treated reagents.

Thiol-Blocking by Nitroxyl (HNO)

³⁵S-Cys (˜5 nM) in phosphate buffer pH 7 and PS-MPTS NPs (1 mg/mL) dispersed in the same buffer were separately mixed with excess Angeli's salt (˜2 mM). Then they were mixed to follow the scintillation inhibition due to binding inhibition. Thiol blocking on both reagents (³⁵S-Cys and PS-MPTS NPs) as well as only one of them were tested. In the HNO concentration-dependent experiment, only the thiols on ³⁵S-Cys (˜1 nM) were blocked.

Thiol Generation by SBH

Sample preparation was performed similar to thiol-blocking by NEM. ³⁵S-Cys (˜2.5 nM) in phosphate buffer pH 7 and PS-MPTS NPs (1 mg/mL) dispersed in the same buffer were separately mixed with excess NEM (˜30 mM) in order to block the thiols. Then the thiol-blocked reagents (both ³⁵S-Cys and PS-MPTS NPs) were mixed to follow the scintillation inhibition due to binding inhibition. A control sample was also prepared without NEM to observe the scintillation due to the specific binding of ³⁵S-Cys to PS-MPTS. After reading SPA, excess SBH (˜15 mM) was added to the sample with NEM, to specifically cleave disulfide bonds of ³⁵S-CySS and reduce it to more thiols and observe scintillation due to new bonds formed between NPs and newly reduced ³⁵S-Cys.

Disulfide Generation by Metals

³⁵S-Cys (2.5 nM) was treated with few representative metals (100 ppm) prior to addition to 1.5 mL PS-MPTS NPs (1 mg/mL). Then ³⁵S-Cys was added to NPs to read scintillation response due to binding. Thiol-disulfide ratio is the key factor in scintillation response, because scintillation occurs on PS-MPTS NPs only due to binding. A set of control samples was prepared with the addition of the same metal-treated ³⁵S-Cys samples to 1 mL LSC. Scintillation response in LSC is not dependent on binding and should stay the same for any thiol-disulfide ratio.

Characterization of Thiol-Functionalized NPs

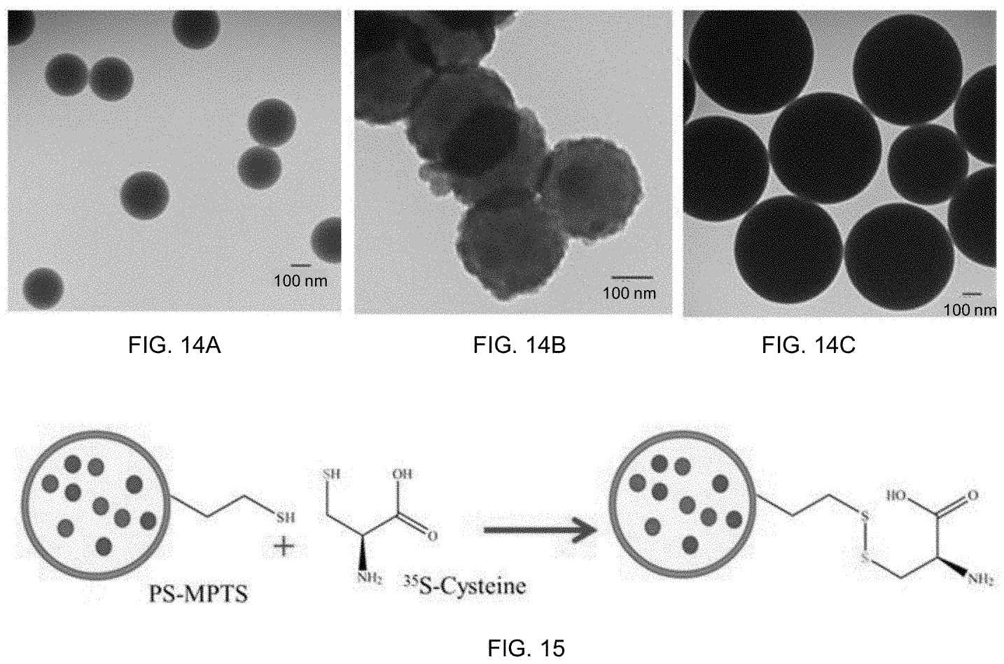

TEM was performed on PS NPs before and after silica coating to measure their size and explore the surface morphology of the NPs. FIGS. 14A-14C show TEM images of PS core, PS-MPTS core-shell, and PS-APTS core-shell NPs. PS-MPTS NPs are raspberry-like NPs with thiol functionality on the surface. PS-APTS NPs that are amine-functionalized look smooth on the surface.

Disulfide Binding of ³⁵S-Cys to PS-MPTS NPs

To illustrate that thiol-functionalized NPs (PS-MPTS) could be used for SPA-based detection of ³⁵S-labeled analytes, the binding of a model compound, ³⁵S-Cys, was examined and the scintillation was quantified. Cys has a thiol group which can bind to another thiol from other molecules to form a disulfide bond. This covalent bond is stable unless a reducing agent is added. In order to investigate the binding of ³⁵S-Cys to thiol-functionalized NPs (PS-MPTS), a binding experiment was performed. FIG. 15 shows the binding scheme of ³⁵S-Cys to PS-MPTS NPs. The scintillation cascade started upon binding.

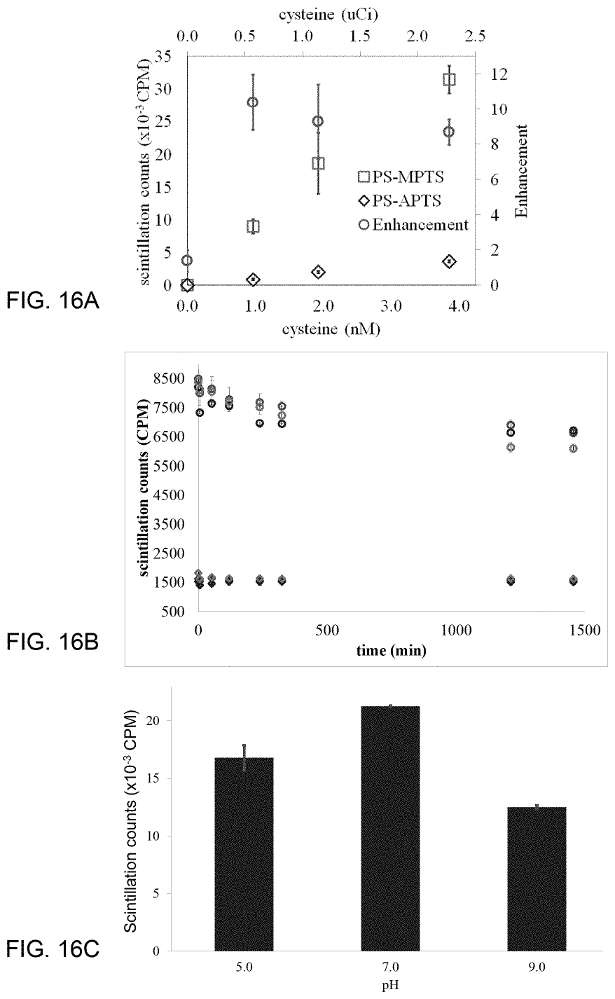

FIG. 16A shows the scintillation counts as a function of the concentration of added ³⁵S-Cys to PS-MPTS and PS-APTS NPs. There is an order of magnitude enhancement in scintillation response observed for PS-MPTS, compared with PS-APTS NPs, which is a strong evidence for specific binding of ³⁵S-Cys to the thiol functionalized NPs. The background scintillation counts observed for PS-APTS NPs is due to the non-proximity effect (the energy transfer from decaying ³⁵S-Cys molecules that are in close proximity to PS-APTS NPs).

Disulfide Exchange/Cleavage

In order to test for the reversibility of disulfide binding on PS-MPTS NPs, disulfide exchange/cleavage was investigated. Disulfide bonds formed between ³⁵S-Cysand PS-MPTS can be cleaved by reducing agents, such as TCEP and DTT. TCEP and DTT were both used as reducing agents because they have different reducing potential and chemistry. Also, disulfide exchange using unlabeled Cys can dynamically displace bound radiolabeled Cys molecules from the surface of the PS-MPTS NPs, which decreases the scintillation intensity.

The circles in FIG. 16B present the time-dependent displacement of bound ³⁵S-Cysfrom the surface of PS-MPTS NPs in the presence of excess TCEP, DTT, and unlabeled Cys, which is a good evidence of the reversibility of disulfide bond formation on the NPs. The decrease in the intensity of scintillation with time, after adding the reducing agents or unlabeled Cys, confirms the specific binding of ³⁵S-Cys to PS-MPTS. The impact of TCEP and DTT and also unlabeled Cys in displacing the ³⁵S-Cys from the surface of the PS-MPTS NPs is similar at early times after addition (i.e. ˜100 min). However, they show different displacement impacts with time. DTT presents higher rate of decrease in scintillation by reducing the disulfide bonds formed between ³⁵S-Cys and PS-MPTS NPs. This confirms the higher reducing activity of DTT compared with TCEP. The diamonds show the impact of TCEP, DTT, and cysteine on scintillation counts on PS-APTS NPs due to non-proximity effect. As one can see the non-proximity effect stays constant, because there is no disulfide exchange on the surface of these NPs.

pH-Dependent Binding

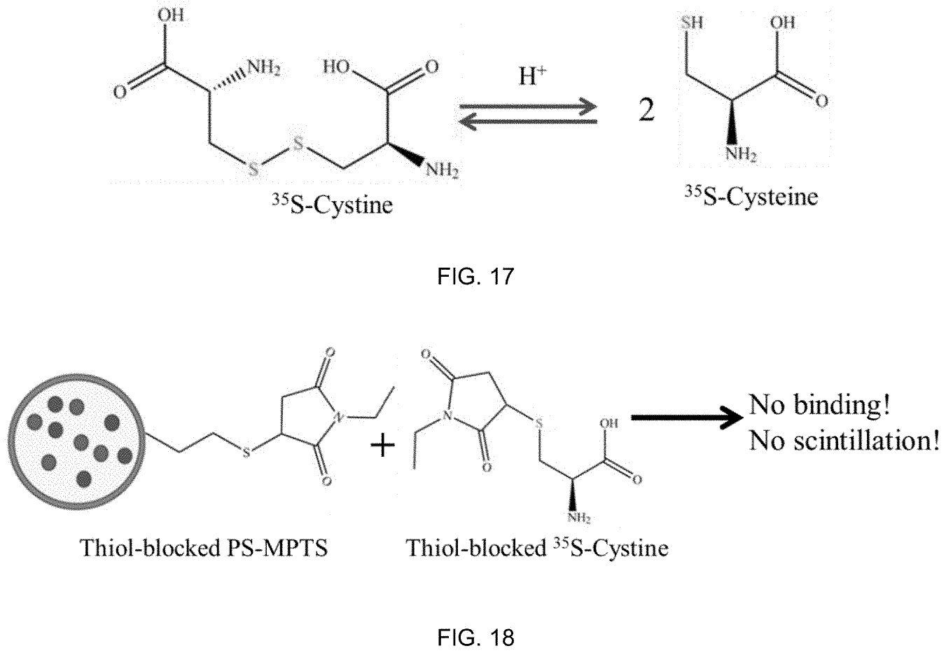

CySS is almost 40% of total Cys equivalent in plasma and Cys is known to undergo a pH-dependent dimerization to CySS. The Binding of ³⁵S-Cys to PS-MPTS NPs at different pH values was examined to evaluate the effect of pH on scintillation response. The binding affinity is a function of the fraction of amino acid in its active monomer form and that is a function of pH as shown in FIG. 17.

Data presented in FIG. 16C confirm that the ratio of monomer to dimer is changing with pH. Interestingly at low pH (pH=5) the binding is lower than neutral pH (pH=7), which is explained by higher concentration of protonated thiol groups on the amino acid without binding ability to NPs. At pH 7, which is closer to pKa of thiol group on Cys (pKa-8.3), a greater fraction of the amino acid is deprotonated (thiolate ion) which forms disulfide bonds with PS-MPTS NPs. Finally, at a higher pH (pH=9) the disulfide form of the amino acid is dominant and consequently binding decreases. This experiment shows that PS-MPTS NPs have a higher binding affinity to ³⁵S-Cysat physiological pH. The pKa of thiol groups on PS-MPTS NPs is about 10.

Thiol-Blocking by NEM Inhibits Binding of ³⁵S-Cysteine to PS-MPTS NPs.

NEM is a highly reactive cyclic alkene, which can react with thiols as an alkylating agent to form a thioether. This N-substituted maleimide is commonly used in protein chemistry as a thiol-blocking reagent, which benefits from its small size. In this experiment, NEM is used to block the thiol groups on both PS-MPTS NPs and ³⁵S-Cys.

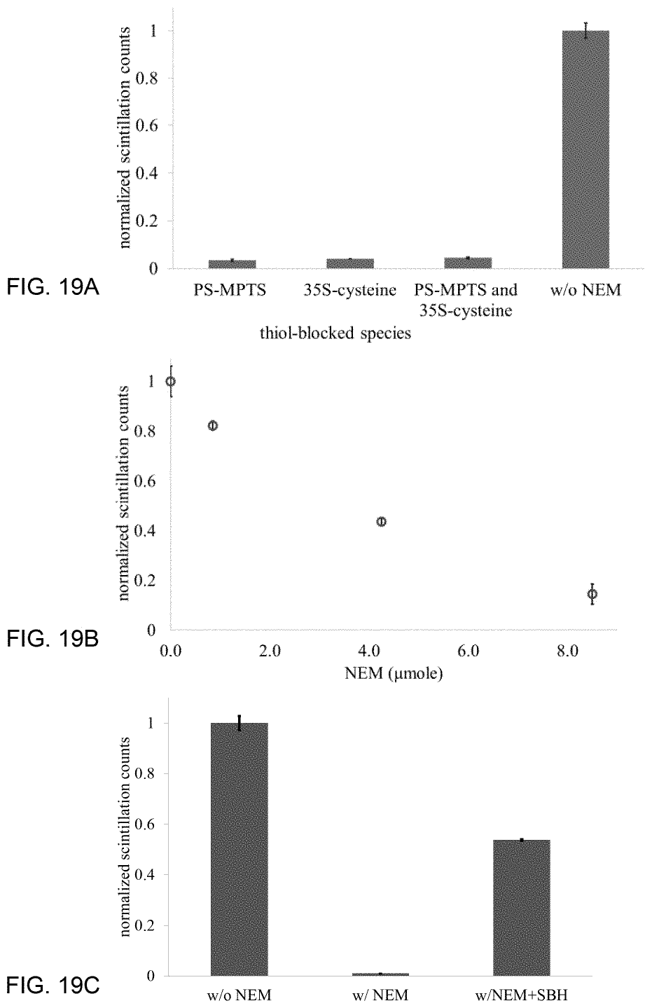

There is no specific binding expected from thiol-blocked reagents (FIG. 18) and hence no significant scintillation should be seen. The scintillation counts measured for the sample with and without NEM is shown in FIG. 19A. The scintillation response observed for the NEM-treated reagents (³⁵S-Cys, PS-MPTS NPs, or both) was indistinguishable from non-proximity effect and a high scintillation is measured for the control sample (with no NEM treatment). Scintillation responses for all samples are normalized to the control sample (without NEM treatment).

The data illustrates successful blocking of thiol groups on ³⁵S-Cys and PS-MPTS NPs, which is another strong evidence for specific binding of ³⁵S-Cys to PS-MPTS NPs via disulfide binding. Thiol blocking by NEM was also performed on a fixed concentration of ³⁵S-Cys by adding different concentrations of NEM. More binding inhibition was observed with higher concentration of NEM, as shown in FIG. 19B. This experiment confirms the capability of PS-MPTS NPs for indirect measurement of thiol-reactive agents.

Thiol Generation by Reduction of Disulfides Using SBH

NEM can be used to block the thiol groups on ³⁵S-Cys to inhibit binding to PS-MPTS NPs, as explained above. In order to measure the remaining part of the amino acids that are in disulfide form, a reducing agent can be used to cleave the disulfide bonds and create new thiols to bind to the NPs. SBH (NaBH₄) is a reducing agent that can cleave the disulfide bonds of CySS and create new Cys molecules. It also inactivates any excess blocking agent (NEM) which prevents blocking of newly formed thiols and thus allows for new bindings to the NPs. This experiment was performed for ³⁵S-Cys at pH 7 on PS-MPTS NPs. The first column of FIG. 19C shows the control sample without NEM treatment. Scintillation response was observed for this sample as a result of specific binding. NEM inhibits binding of ³⁵S-Cys to PS-MPTS (2^{nd} column). Once SBH was added to the second sample, new binding and scintillation was observed, which can be attributed to the thiols released from ³⁵S-CySS (represented in the 3^{rd} column). These results show almost one third of the amino acid was in its disulfide form (CySS). Scintillation responses for the samples are normalized to the control sample (without NEM treatment). This experiment confirms that the NPs of the present invention can be used to measure thiols and disulfides in a mixture. This is helpful to evaluate the thiol-disulfide ratio of the mixtures that represents the redox status.

Thiol-Blocking by Nitroxyl (HNO) Partially Inhibits Binding of ³⁵S-Cysteine to PS-MPTS NPs.

Angeli's salt decomposes to variety of nitrogen oxide species depending on pH. Nitric oxide (NO) is the dominant product of the decomposition of Angeli's salt at a pH less than 4. HNO is produced in the pH range 4-8 which then dimerizes and decomposes to nitrous oxide (N₂O). The protonation of N in Angeli's salt produces HNO which has shown promising characteristics as a treatment for cardiovascular disorders. HNO is also a potential drug for breast cancer and is used as a treatment for alcoholism. HNO produced from Angeli's salt decomposition is a thiophil which rapidly reacts with cysteine and blocks thiols to yield sulfinamide, as shown in the following scheme:

$${ONNOO}^{2 -} + {H}^{+} • HNO + {NO}_{{2}^{-}}$$

$$HNO + RSH • {RSONH}_{2}$$

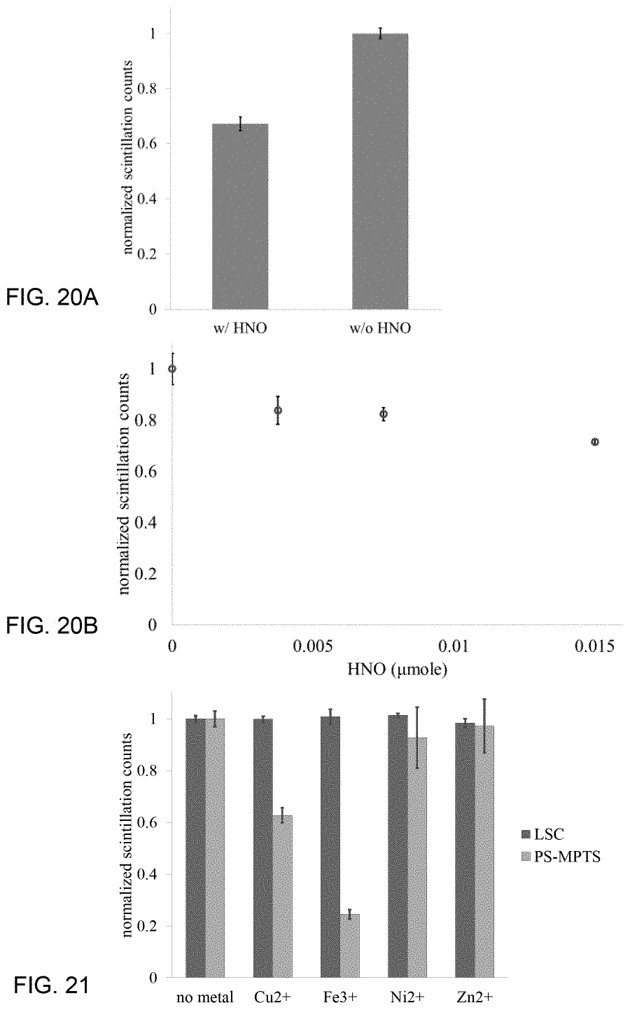

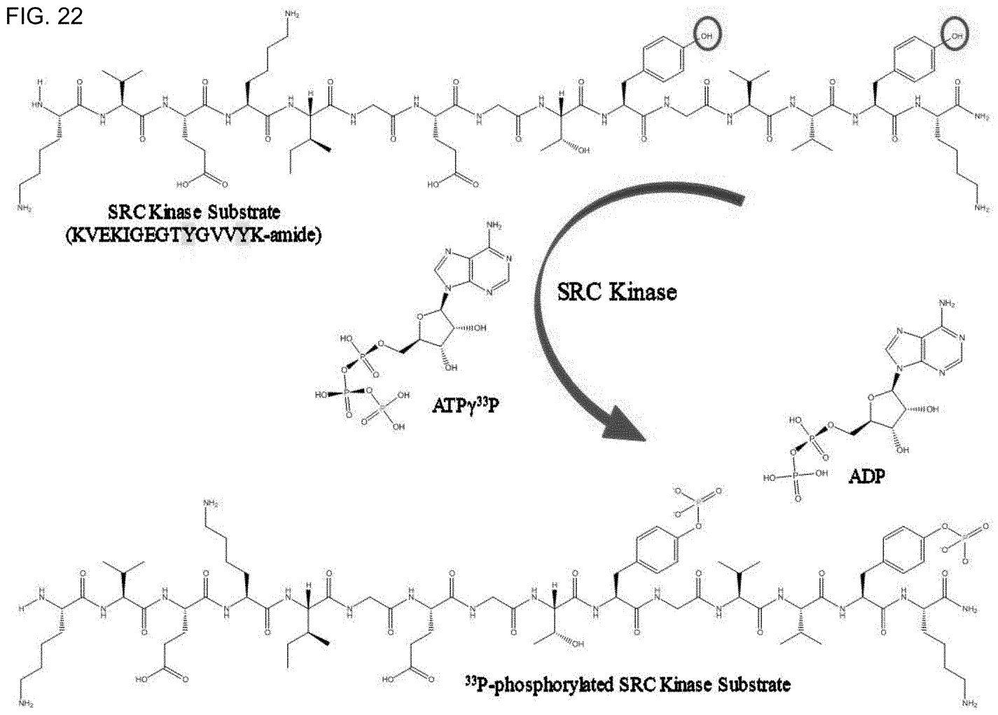

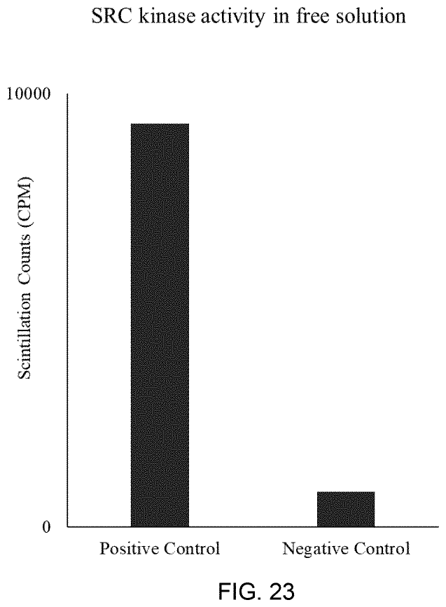

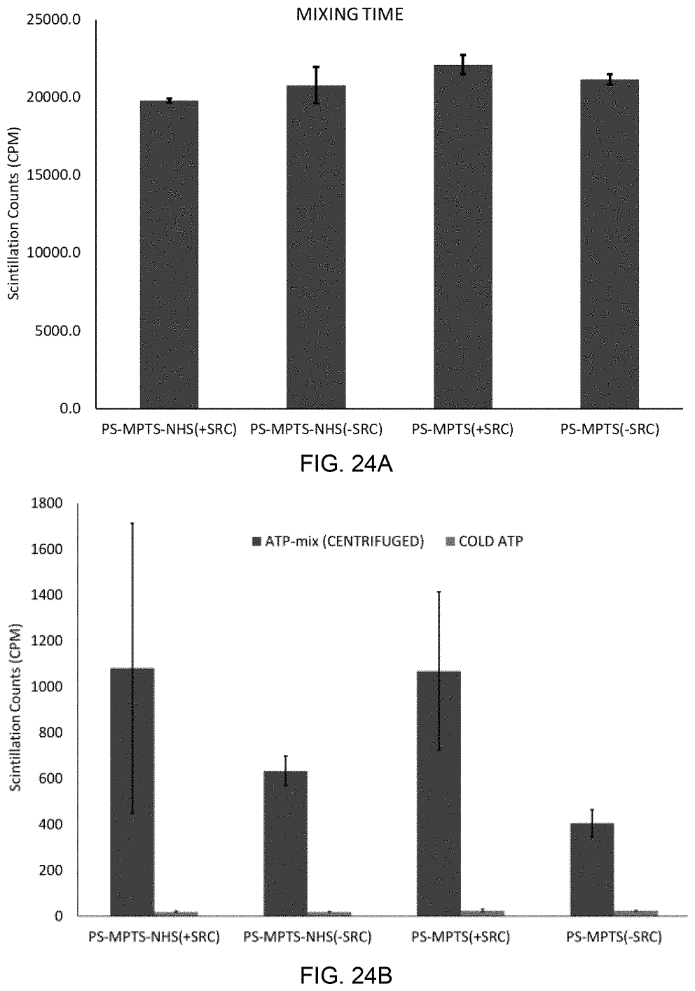

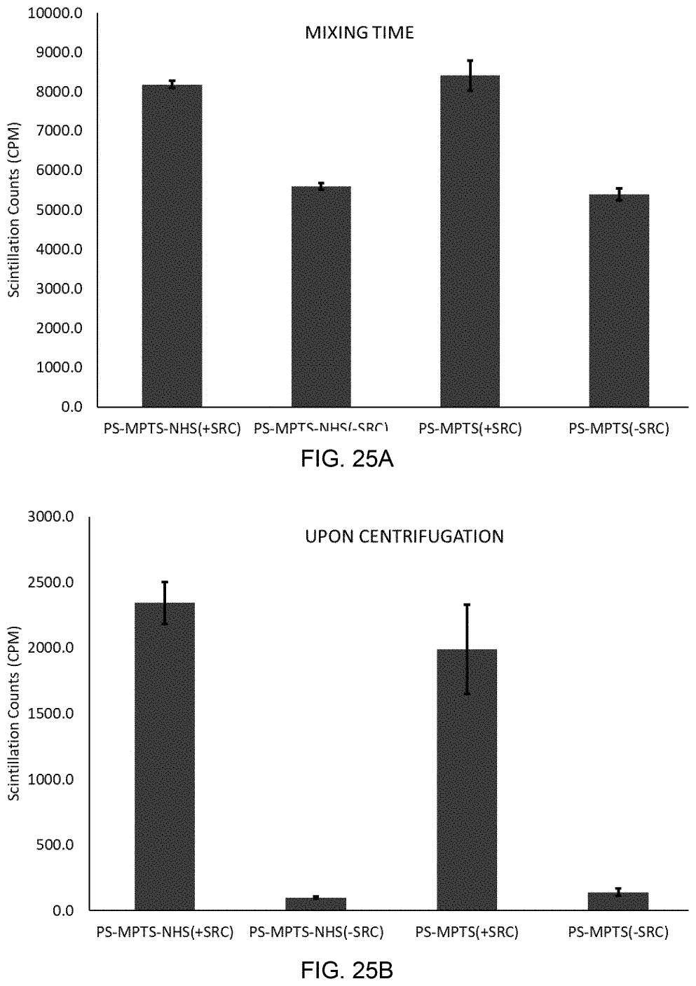

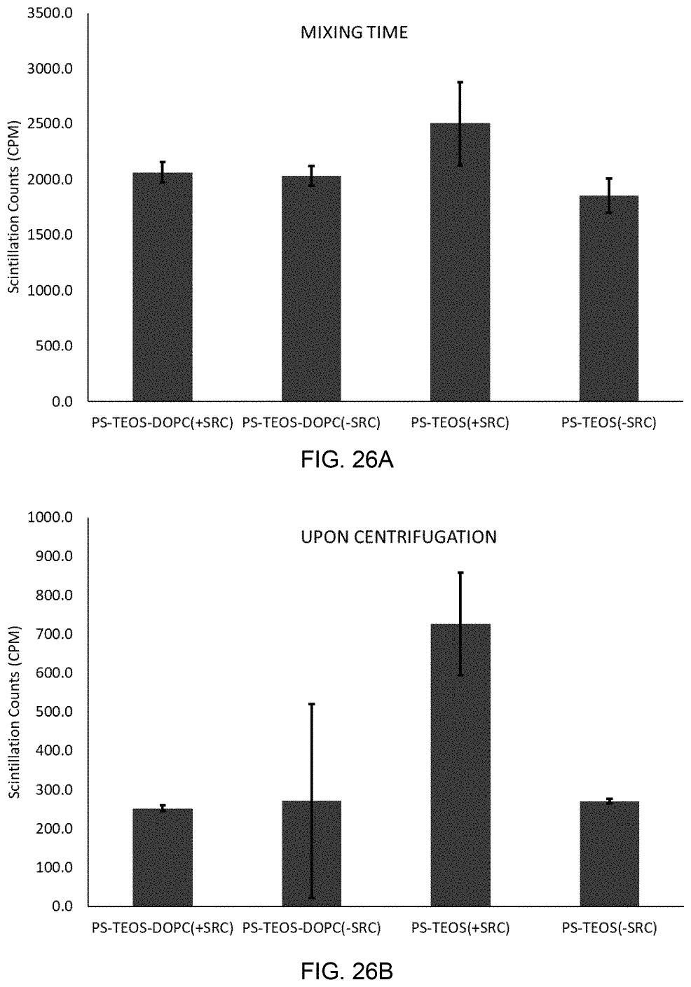

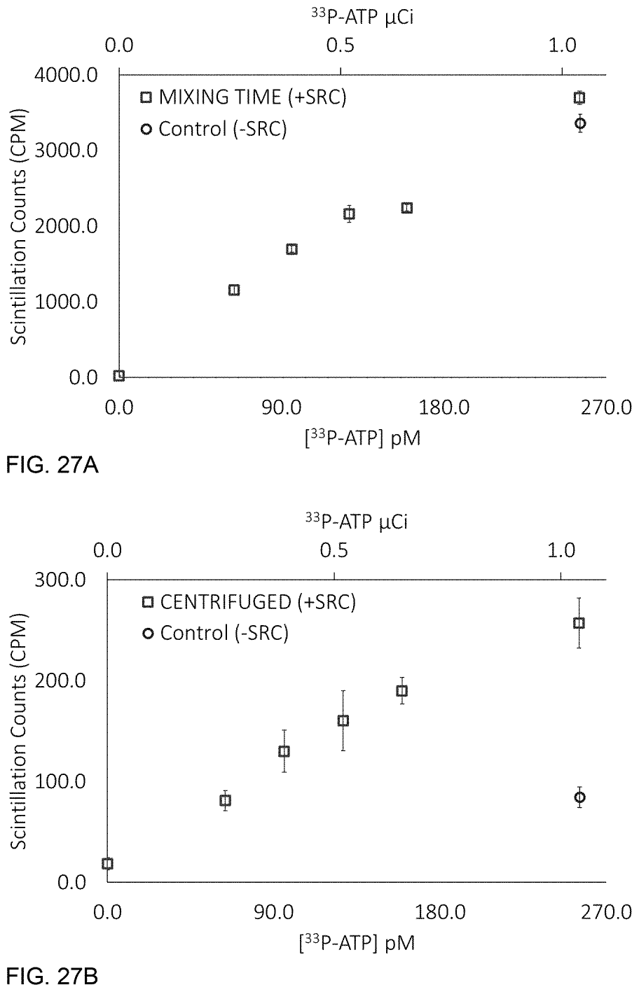

Therefore, HNO can be indirectly measured with the biosensor presented in this report by blocking the thiols and following the decrease in scintillation response as a function of HNO concentration. Similar to NEM treatment, both ³⁵S-Cys and PS-MPTS NPs were treated with Angeli's salt separately to block all thiol groups, and then mixed to check for the inhibition of specific binding. Disulfide bond formation was inhibited due to the formation of sulfinamide. However, FIGS. 20A-20B show binding of ³⁵S-Cys was only partial (˜30%). This may be explained by the reaction pathway, which occurs in two steps. In the first step, the nucleophilic reaction of thiol with the nitrogen on HNO (electrophile) produces hydroxysulfenamide. This compound undergoes two different reaction pathways in the second step. It either produces sulfonamide, which is the thiol blocked reagent, or reacts with other thiols to produce disulfides and hydroxylamine. Therefore, more thiols are available through the second reaction pathway which facilitates binding and scintillation response as opposed to NEM-treatment which completely blocks the thiols.

Thiol blocking by HNO was also performed on a fixed concentration of ³⁵S-Cys by adding different concentrations of Angeli's salt. More binding inhibition was observed with higher concentration of HNO that is the product of Angeli's salt decomposition, as shown in FIG. 20B. However, the lowest scintillation response observed in the highest concentration of HNO is around 70%, which is consistently representative of 30% binding inhibition. Scintillation responses for the samples are normalized to the sample without HNO treatment. Although partial binding inhibition is observed for HNO, this experiment is still a strong evidence for the possibility of using PS-MPTS NPs for indirect measurement of HNO.

Disulfide Generation Using Oxidative Metals

Transition metals can oxidize amino acids via Fenton reaction by forming hydroxyl radicals.¹⁰Representative metals were used in this work to investigate the oxidation of thiol groups of ³⁵S-Cys to disulfides. Consequently, the scintillation intensity decreased due to the decreased binding of ³⁵S-Cys to PS-MPTS NPs. In the control samples that were prepared by LSC, no difference was expected between the vials with no metals and those with added metals because the scintillation in LSC does not occur as a result of binding. Therefore, a constant intensity of scintillation is expected from LSC control samples due to the same amount of radiolabeled amino acid (monomer or dimer). FIG. 21 shows the result of this experiment. In control samples made with LSC, there is no difference between the sample with no metals and the rest of the samples with added metals. However, in the case of the samples prepared using the PS-MPTS NPs, different scintillation intensities are observed due to the difference in oxidation state of the mixture. The oxidizing ability of metals is different and iron has the highest oxidative activity among these four metals. This trend is consistent with the trend of oxidizing potential of the presented metals (Fe³⁺+0.771, Cu²⁺+0.337, Ni²⁺−0.25, and Zn²⁺−0.763 V vs. standard hydrogen electrode (SHE)). In each set of data (PS-MPTS NPs or LSC), scintillation response for each sample was normalized to the sample without metals.

NanoSPA is a potentially strong biosensor candidate for real-time intracellular analyses because of the advantages discussed above. Feeding cells with radiolabeled analyte of interest leads to selective analysis in a pool of molecules with similar structures and properties. The present invention advantageously provides for a selective and specific biosensor for ³⁵S-Cys which can detect this amino acid in a concentration range as low as sub-nanomolar at physiological pH and does not require any light source. Specific binding of ³⁵S-Cys to the NPs occurs in seconds and can be useful for intracellular studies of thiol containing compounds. PS-MPTS NPs may also be potential biosensors for other biothiols such as hCys. These NPs may also be used for indirect measurement of thiol-reactive agents.

### Example 6

The following is a non-limiting example of scintillating core-shell nanoparticles as turn-on nanosensors for selective detection of ³³P, a lower energy phosphorus isotope, to analyze kinase activity. Equivalents or substitutes are within the scope of the present invention.

Kinases are enzymes that catalyze phosphate transfer from adenosine triphosphate (ATP) to their target substrates to produce adenosine diphosphate (ADP) and phosphorylated substrates. Phosphates play important roles in transfer of energy (ATP) and information (DNA and RNA) in biology and phosphorylation is one of the most important post translational modifications which contribute to cell signaling cascades. Referring to FIG. 22, phosphorylation of peptide/protein substrates occurs on a hydroxyl group on an amino acid residue and based on the phosphorylated amino acid residue, kinases are classified as serine/threonine or tyrosine kinases.SRC (sarcoma) kinase is a tyrosine kinase and catalyzes phosphate transfer from ATP to a tyrosine residue in its peptide substrate. SRC kinase plays key roles in cell motility, morphology, differentiation, proliferation, and bone resorption. Overexpression or increased specific activity of SRC kinase is observed in many types of human cancers, such as breast, colon, lung, and ovarian cancer, and SRC kinase inhibitors such as SRC Inhibitor-1 are investigated as potential treatment of malignancies.

Various techniques are used to study kinases due to their importance as therapeutic targets for cancer treatment. Enzyme-linked immunosorbent assay (ELISA) requires multiple expensive reagents (e.g. antibodies) and many washing steps. Therefore, ELISA is a costly and low throughput technique for kinase studies. Fluorescence based assays are useful for kinase studies, but they are susceptible to interference and artifacts. Radioactive assays require radiolabeled materials but they are robust and less prone to artifacts as compared to fluorescence based assays and provide a better indicator of kinase activity. ATPγ³³P is used in radioactive assays for kinase studies with radioisotope ³³P on the γ-phosphate group that is transferred to the substrate of a kinase. LSA is used in phosphocellulose filter paper-based kinase studies and involves many washing steps that make it tedious and not environmentally friendly due to the high volume of radioactive and toxic LSC waste. SPA microbeads are used for separation-free analysis of kinases. SPA works based on the interaction of ionizing beta particles emitted from decaying ³³P-radioisotope with scintillating fluorophores that are doped into SPA beads. The commercial SPA beads are dense and large (at least 1 μm) which makes them hard to disperse in samples. Additionally, SPA microbeads require specific coupling functionalities on the kinase substrates and on the beads, which adds to the cost and complexity of the analysis.

The nanoparticles of the present invention provide a cost-effective and high throughput technique for kinase studies. Polystyrene-silica core-shell nanoparticles with reporter fluorophores doped in the polystyrene core were fabricated to perform nanoSPA. The core-shell NPs are hydrophilic and easy to disperse in aqueous samples, as opposed to SPA microbeads. More importantly, nanoSPA is a separation-free or homogeneous technique, as opposed to other techniques such as ELISA. Only radiolabeled molecules bound to nanoSPA (i.e. ³³P-phosphorylated substrate) generate a significant signal. Therefore, it is unnecessary to separate excess unbound radiolabeled reagents (i.e. ATPγ³³P) from the mixture of NPs because the radioactive energy from unbound ATPγ³³P dissipates in the solution before it reaches the NPs. The homogeneity of nanoSPA minimizes the waste disposal and complexity of the analysis, which is beneficial for high throughput screening of therapeutics. The analysis based on nanoSPA is performed by combining the kinase mixture (substrate+ATPγ³³P±kinase) with NPs and measuring the scintillation signal, which makes this technique simple, robust, and easy to automate. Moreover, nanoSPA is useful for intracellular analysis of kinases due to the smaller size of the NPs as compared to commercial SPA microbeads. In some embodiments, NPs may be incorporated in live cells that are fed with ATPγ³³P for intracellular study of kinases by optical imaging.

Materials and Methods

ATPγ³³P was purchased from American Radiolabeled Chemicals Inc. (St. Louis, Mo.). 1,2-dioleoyl-sn-glycero-3-phosphocholine (DOPC) lipid was purchased from Avanti Polar Lipids, Inc. (Alabaster, Ala.). SRC kinase kit (kinase, substrate, buffer, DTT, and MnCl₂) was purchased from Promega Corporation (Madison, Wis.). TEOS (98%), APTS (99%), MPTS (97%), MPH (97%) and sodium hydrogen phosphate (99%) were purchased from Sigma Aldrich (St. Louis, Mo.). Styrene (99%), pTP (99%), and DMPOPOP (98%) were purchased from ACROS Organics (Geel, Belgium). ACS-grade isopropyl alcohol was purchased from Merck (Kenilworth, N.J.). Biocount LSC was obtained from Research Product International (Mt. Prospect, Ill.). ACS-grade ammonium hydroxide was purchased from EMD Millipore (Billerica, Mass.). Sodium hydroxide (98.9%) was purchased from Avantor Performance Materials (Center Valley, Pa.). Purified water (18 MΩ) was obtained using Easy Pure UV/UF Barnstead. A Beckman Coulter LS 6000IC was used for scintillation measurements. A Tecnai G2 Spirit 20-120 kV transmission electron microscope (TEM) was used to characterize the NPs.

NanoSPA Particle Fabrication and Characterization

PS core NPs doped with scintillant fluorophores were prepared in three steps. First, inhibitors were removed from styrene by passing 3 g of styrene through an alumina column. Second, styrene was polymerized using a surfactant-free emulsion polymerization method. 3 g styrene was dispersed in 50 mL degassed nanopure water by stirring. Polymerization was initiated at 80° C. using AAPH. The emulsion was stirred continuously for 12 hrs. Excess styrene was removed from the flask by evaporation at reduced pressure. The concentration of PS NPs was obtained by freeze-drying a known volume of the NP solution and weighing the dry NPs. Third, primary and secondary scintillants, pTP (1.14 mmoles) and DMPOPOP (0.135 mmoles) dissolved in 20 mL of a 90:10 mixture of chloroform and isopropyl alcohol, were used for ex-situ doping of NPs by swelling the PS core. The organic mixture of scintillants was added to 500 mg PS NPs dispersed in 100 mL water and sonicated and stirred continuously for 2 hours. Excess organic phase was removed from the mixture by evaporation at reduced pressure.

The scintillant-doped PS core NPs were then coated with silica shells to obtain silica-coated (PS-TEOS) or thiol-functionalized (PS-MPTS) core-shell NPs. 110 mg PS core NPs were dispersed in a mixture of 200 mL isopropanol and 40 mL water. 7 mL NH₄OH was added to adjust the pH of the mixture. Then 2 mL TEOS or 2 mL of a mixture of 90% (v/v) TEOS and 10% (v/v) MPTS was added dropwise to the PS NPs to obtain PS-TEOS or PS-MPTS, respectively. The mixture was stirred for an hour and then the core-shell NPs were obtained after centrifugation and rinsing with water.

PS-MPTS NPs were tested for non-specific adsorption of ³³P-phosphorylated SRC kinase substrate by electrostatic attraction of positively charged SRC kinase substrates to negatively charged silane groups on nanoSPA. To specifically bind ³³P-phosphorylated SRC kinase substrate to nanoSPA, PS-MPTS NPs were further functionalized with a cross linker (Mal-PEG2-NHS ester) by thiol-ene coupling of the maleimide end group of the linker to thiol groups on the surface of PS-MPTS NPs in citrate buffer (pH 6.5) for 30 minutes, to obtain PS-MPTS-NHS NPs. The NHS end of the linker on PS-MPTS-NHS NPs was used to facilitate peptide coupling of SRC kinase substrate through the amine groups on its lysine amino acid residues.

PS-TEOS NPs were coated with DOPC lipid to illustrate the minimization of non-specific adsorption of ³³P-phosphorylated SRC kinase substrate to lipid-coated nanoSPA. Lipid coating was done by using 6 mg DOPC for 6 mg PS core NPs (6 samples). 6 mg DOPC (250 μL, 25 μg/μL) was obtained from the freezer and dried with argon to remove excess chloroform. The residual chloroform was removed by lyophilizing for 4 hours. 3 mL nanopore water was added to the dried DOPC and mixed. The multilamellar vesicles of DOPC that was formed in the suspension of DOPC in water were broken into vesicles by sonication of the lipid suspension in a glass vial. Sonication was performed for 10 minutes and stopped for 10 minutes to avoid heating the sample. The sonication/rest cycle was repeated four times. The suspension of DOPC vesicles was divided into 6 plastic scintillation vials and 500 μL DOPC (2 mg/mL) was obtained in each vial. 1 mg PS-TEOS NPs in 25 μL water was added to each vial to preserve the concentration of DOPC. The mixture of PS-TEOS NPs and DOPC vesicles was incubated at room temperature overnight (at least 12 hours) to obtain lipid-coated NPs (PS-TEOS-DOPC) after centrifugation for 15 minutes at 3000 rpm. There was no rinsing step or sonication after the NPs were collected.

Analysis of SRC Kinase Activity

SRC kinase was used to phosphorylate its peptide substrate (KVEKIGEGTYGVVYK-amide) with ATPγ³³P or a mixture of ATP and ATPγ³³P. All samples were prepared in SRC kinase buffer (pH 7). Kinase activity analyses by nanoSPA were performed by combining a pre-incubated kinase mixture (substrate+ATPγ³³P ±kinase) with NPs and measuring the scintillation signal using LSC instrument. 1 mL NPs (1 mg core/mL) was used for all samples in nanoSPA experiments.

Analysis of SRC Kinase Activity in Free Solution

ATP-mix was prepared by combining 1 μL ATPγ³³P (8.72 ρCi) with 20 μL 0.13 mM ATP. A mixture of 4 μL 2 μg/μL SRC kinase and 10 μL 0.1 μg/μL SRC kinase substrate (KVEKIGEGTYGVVYK-amide) was prepared. Then 10 μL ATP-mix was added to the kinase mixture (24 μL total volume). A negative control sample was prepared using the mixture of SRC kinase substrate and ATP-mix, with no SRC kinase enzyme. 4 μL buffer was added to the negative control sample to compensate for the volume. Table 1 shows the amount of kinase mixture components for positive and negative control samples. Kinase mixtures were incubated at room temperature for 30 minutes for phosphorylation reaction on SRC kinase substrate. Phosphocellulose filter paper-based LSA was performed for measurement of phosphorylation. The kinase mixtures were deposited on filter papers and washed three times with 0.85% phosphoric acid and once with acetone to remove excess ATPγ³³P. The filter papers were submerged in 3 mL LSC to monitor the scintillation due to immobilized ³³P-phosphorylated SRC kinase substrates on the filter paper.

TABLE 1
Samples prepared for SRC kinase activity evaluation in free
solution by LSA.

| | | | ATPγ³³P | SRC |
|---|---|---|---|---|
| | SRC kinase | [ATP] | activity | kinase |
| Sample ID | substrate (μg) | (μM) | (μCi) | (ng) |
| Positive Control | 1 | 50.0 | 4.15 | 8 |
| Negative Control | 1 | 50.0 | 4.15 | 0 |

Referring to FIG. 23, scintillation counts were collected by LSC. The significant difference observed for the samples confirmed the activity of SRC kinase. The residual scintillation observed for the negative control sample is due to adsorption of ATPγ³³P molecules to the filter papers which were not completely removed in the washing steps with phosphoric acid and acetone.

Analysis of SRC Kinase Activity by nanoSPA (Specific Binding and Non-Specific Adsorption)

Kinase mixtures were prepared in a set of separate experiments, see Table 2, using ATP-mix (Hot-Cold ATP), ATP (Cold ATP), and ATPγ³³P (Hot ATP), as described above. ATP-mix was prepared for Hot-Cold ATP samples by combining 6 μL ATPγ³³P (50.91 μCi) with 114 μL 0.13 mM ATP. The kinase mixtures were incubated for 30 minutes in case of Cold ATP and Hot-Cold ATP samples. However, Hot ATP samples were incubated overnight because of much lower concentration of ATP to provide enough time for the phosphorylation reaction.

To evaluate SRC kinase activity and the sensitivity of nanoSPA to ATP, thiol-functionalized NPs, doped with pTP and DMPOPOP, were fabricated and used to immobilize ³³P-phosphorylated SRC substrate on the surface of nanoSPA. NanoSPA functionalized with a sulfhydryl-to-amine crosslinker, i.e. PS-MPTS-NHS, was used for specific binding of ³³P-phosphorylated SRC substrate. NanoSPA without the sulfhydryl-to-amine crosslinker, PS-MPTS, was used for non-specific adsorption of ³³P-phosphorylated SRC kinase substrates to the surface of NPs.

TABLE 2
Samples prepared for SRC kinase activity evaluation by nanoSPA.

| | | SRC | | | |
|---|---|---|---|---|---|
| | | kinase | | ATPγ³³P | SRC |
| | | substrate | | activity | kinase |
| | NanoSPA Sample ID | (μg) | [ATP] | (μCi) | (ng) |
| Hot- | PS-MPTS-NHSNPs | 1 | 50.0 μM | 4.24 | 10 |
| Cold | (+SRC) | | | | |
| ATP | PS-MPTS-NHSNPs | 1 | 50.0 | 4.24 | 0 |
| | (−SRC) | | | | |
| | PS-MPTS NPs(+SRC) | 1 | 50.0 | 4.24 | 10 |
| | PS-MPTS NPs (−SRC) | 1 | 50.0 | 4.24 | 0 |
| Cold | PS-MPTS-NHS NPs | 1 | 50.0 μM | 0 | 10 |
| ATP | (+SRC) | | | | |
| | PS-MPTS-NHS NPs | 1 | 50.0 | 0 | 0 |
| | (−SRC) | | | | |
| | PS-MPTS NPs (+SRC) | 1 | 50.0 | 0 | 10 |
| | PS-MPTS NPs (−SRC) | 1 | 50.0 | 0 | 0 |
| Hot | PS-MPTS-NHS NPs | 1 | 3.62 pM | 1.30 | 10 |
| ATP | (+SRC) | | | | |
| | PS-MPTS-NHS NPs | 1 | 3.62 | 1.30 | 0 |
| | (−SRC) | | | | |
| | PS-MPTS NPs (+SRC) | 1 | 3.62 | 1.30 | 10 |
| | PS-MPTS NPs (−SRC) | 1 | 3.62 | 1.30 | 0 |

FIGS. 24A-24B shows scintillation data from immobilization of ³³P-phosphorylated SRC substrate, using ATP-mix, on nanoSPA. In each pair of samples (NPs with linker, PS-MPTS-NHS, or NPs without linker, PS-MPTS) there is a positive (+SRC: SRC kinase is added to ³³P-phosphorylate the substrate) and a negative control sample (−SRC: no kinase was added). FIG. 24A illustrates scintillation counts upon mixing NPs with kinase mixtures. There is no significant difference between the samples due to high non-proximity effect by excess ATPγ³³P. Samples were centrifuged and the supernatant was replaced by buffer to minimize the non-proximity effect, as shown in FIG. 24B. The decrease in signal by centrifugation is because the ATP-mix used for the phosphorylation mostly contained ATP and ATPγ³³P/total ATP percentage was 2.4×10⁻⁵%. Due to much higher concentration of ATP as compared to ATPγ³³P phosphorylation occurred mostly by ATP and the signal/background ratio was poor (2-3). In FIG. 24B, the results of kinase analysis using cold ATP is also included as a comparison to the extent of scintillation on nanoSPA by using ATP-mix in the phosphorylation reaction, as described.

To improve the signal to background ratio, a similar experiment was performed using only ATPγ³³P. The kinase mixtures were prepared by mixing ATPγ³³P, SRC kinase substrate, and with or without SRC kinase. Incubation of kinase mixtures overnight facilitated phosphorylation over a longer time to compensate for the low concentration of ATPγ³³P. FIGS. 25A-25B show scintillation data from immobilization of ³³P-phosphorylated SRC substrate, using ATPγ³³P, on nanoSPA. FIG. 25A shows scintillation data collected upon mixing kinase mixtures with NPs. There is a significant difference between samples with and without SRC kinase, which confirms the kinase activity. However, samples were centrifuged and the supernatant was replaced with buffer to minimize the non-proximity effect caused by excess ATPγ³³P, as shown in FIG. 256. Although the signal decreased dramatically upon centrifugation, the positive and negative control samples became more distinguishable, which improved signal/background ratio to over an order of magnitude. Scintillation counts on NPs with linker is higher than those without linker, which shows that using a linker to specifically bind the ³³P-phosphorylated SRC kinase substrate to NPs contributes to the scintillation counts. All samples were rinsed several times with buffer, methanol, and Tween®20 to compare the stability of the bound ³³P-phosphorylated SRC kinase substrate on NPs with and without linker. Scintillation counts on NPs without linker were more impacted by the washing steps, as compared to samples prepared by NPs with linker, data not shown. The washing steps also confirmed the specific binding of ³³P-phosphorylated SRC kinase substrate to NPs with linker.

Analysis of SRC Kinase Activity by Lipid-Coated nanoSPA (Inhibition of Non-Specific Adsorption)

Kinase mixtures were prepared using ATPγ³³P, as previously described, and incubated overnight, see Table 3. To demonstrate the reduction of non-specific adsorption of ³³P-phosphorylated SRC kinase substrates to nanoSPA, a set of samples were prepared using lipid-coated NPs, PS-TEOS-DOPC to monitor scintillation. PS-TEOS NPs were used for non-specific adsorption of ³³P-phosphorylated SRC kinase substrates to nanoSPA to compare the scintillation counts on lipid-coated NPs to NPs without lipid coating.

TABLE 3
Samples prepared for SRC kinase activity evaluation by lipid-
coated nanoSPA.

| | | SRC | | | |
|---|---|---|---|---|---|
| | | kinase | | ATPγ³³P | SRC |
| | | substrate | [ATP] | activity | kinase |
| | NanoSPA Sample ID | (μg) | pM | (μCi) | (ng) |
| Hot | PS-TEOS-DOPC NPs | 1 | 1.67 | 0.60 | 10 |
| ATP | (+SRC) | | | | |
| Lipid | PS-TEOS-DOPC NPs | 1 | 1.67 | 0.60 | 0 |
| | (−SRC) | | | | |
| | PS-TEOS NPs (+SRC) | 1 | 1.67 | 0.60 | 10 |
| | PS-TEOS NPs (−SRC) | 1 | 1.67 | 0.60 | 0 |

DOPC coating on the NPs led to decreased scintillation counts by inhibition of non-specific adsorption of ³³P-phosphorylated SRC substrate to the surface of the NPs. FIGS. 26A-26B show scintillation data from immobilization of ³³P-phosphorylated SRC substrate, using ATPγ³³P, on NPs coated with DOPC (PS-TEOS-DOPC) and PS-TEOS. FIG. 26A shows scintillation data collected upon mixing kinase mixtures with NPs which shows a slight decrease in scintillation counts on PS-TEOS-DOPC NPs due to inhibition of non-specific adsorption. FIG. 26B shows scintillation data collected after centrifugation and replacement of supernatant with buffer which demonstrates the inhibition of non-specific adsorption more clearly.

Analysis of SRC Kinase Activity by nanoSPA at Varying Concentrations of ATPγ³³P

To demonstrate the sensitivity of nanoSPA to ATP, kinase mixtures were prepared with constant concentration of SRC kinase and SRC substrate at varying concentrations of ATPγ³³P, see Table 4. PS-MPTS-NHS NPs were used to monitor phosphorylation by specifically binding to ³³P-phosphorylated SRC kinase substrates. A negative control sample was prepared at the highest concentration of ATPγ³³P with no SRC kinase added. Kinase mixtures were incubated overnight to provide enough time for the phosphorylation reaction. These samples were then mixed with PS-MPTS-NHS NPs and the scintillation signal was collected by LSC.

TABLE 4
Samples prepared at varying [ATPγ³³P] for SRC kinase activity
evaluation by nanoSPA.

| | | SRC | | | |
|---|---|---|---|---|---|
| | | kinase | | ATPγ³³P | SRC |
| | | substrate | [ATP] | activity | kinase |
| | NanoSPA Sample ID | (μg) | pM | (μCi) | (ng) |
| Hot | PS-MPTS-NHS NPs (+SRC) | 1 | 0 | 0 | 10 |
| ATP | PS-MPTS-NHS NPs (+SRC) | 1 | 63.8 | 0.26 | 10 |
| | PS-MPTS-NHS NPs (+SRC) | 1 | 95.7 | 0.39 | 10 |
| | PS-MPTS-NHS NPs (+SRC) | 1 | 127.6 | 0.52 | 10 |
| | PS-MPTS-NHS NPs (+SRC) | 1 | 159.5 | 0.65 | 10 |
| | PS-MPTS-NHS NPs (+SRC) | 1 | 255.2 | 1.04 | 10 |
| | PS-MPTS-NHS NPs (−SRC) | 1 | 255.2 | 1.04 | 0 |

FIGS. 27A-27B show scintillation data from immobilization of ³³P-phosphorylated SRC substrate on PS-MPTS-NHS NPs. FIG. 27A shows scintillation data collected upon mixing kinase mixtures with NPs. Scintillation counts increased by increasing ATPγ³³P concentration because more ³³P-phosphorylated SRC substrate was produced during the phosphorylation reaction and more ³³P-phosphorylated SRC substrate was immobilized on the surface of NPs. Similar signal was observed for the positive and negative control samples at the highest ATPγ³³P concentration which is a consequence of non-proximity effect. Therefore, all samples were centrifuged and the supernatants were replaced with buffer to minimize the non-proximity effect by removing excess ATPγ³³P. FIG. 27B shows scintillation data collected upon centrifugation. Scintillation counts for all samples decreased. However, the increasing trend in samples including SRC kinase (positive control samples) was preserved. The negative control sample showed a much smaller scintillation signal after centrifugation.

While the SPA particles of the present invention have been shown to function as sensors for thiol-disulfide measurements and for intracellular analysis of kinases, it is to be understood that these are non-limiting examples, and that SPA may be used in other applications for analyzing various molecular species.

### Example 7

The following are further examples of producing scintillant-doped polystyrene core nanoparticles using the swelling-deswelling process. Equivalents or substitutes are within the scope of the present invention.

Fabrication of Scintillant-Doped Polystyrene Core nPs

Scintillant fluorophore 1 and scintillant fluorophore 2 are dissolved in a solvent. Scintillant fluorophores dissolved in solvent are added directly to the aqueous polystyrene nanoparticle solution in a round-bottomed flask. The nanoparticle solution is sonicated using a bath sonicator for several minutes to disperse solvent droplets throughout the water. A small amount of solvent is then removed under reduced pressure using a rotary evaporator. The nanoparticle solution is sonicated again for several minutes, followed by further solvent removal under reduced pressure. This sonication/solvent removal process is repeated for a total of 5 cycles, or until as much of the organic solvent is removed as possible.

TABLE 5
Examples of Scintillant fluorophore 1 that may be used in the
nanoparticles of Example 7. λₑₓ is the excitation wavelength,
λₑₘ is the emission wavelength.

| Scintillant fluorophore 1 | λₑₓ (nm) | λₑₘ (nm) |
|---|---|---|
| Para-terphenyl (pTP) | 275 | 340 |
| 2,4 diphenyloxazole (PPO) | 300 | 370 |
| 1-phenyl-3-mesityl-2-pyrazoline (PMP) | 355 | 430 |
| naphthalene | 275 | 320 |
| 2-(4-biphenylyl)-5-(4-ter-butylphenyl)- | 300 | 360 |
| 1,3,4-oxadiazole (butyl-PBD) | | |
| anthracene | 370 | 390 |

TABLE 6
Examples of Scintillant fluorophore 2 that may be used in the
nanoparticles of Example 7.

| Scintillant fluorophore 2 | λₑₓ (nm) | λₑₘ (nm) |
|---|---|---|
| 1,4-bis(2-methylstyryl)benzene (bis-MSB) | 340 | 420 |
| 1,4-bis-2-(4-methyl-5-phenyloxazolyl)- | 350 | 430 |
| benzene (DMPOPOP) | | |
| 7H-benzimidazo(2,1-a)benz. | 300 | 480 |
| (de)isoquinoline-7-one (BBQ) | | |
| decacyclene | 380 | 515 |
| rubrene | 530 | 580 |
| tris(1,3-diphenyl-1,3-propanedionato)- | 340 | 615 |
| (1,10-phenanthroline) europium (III) | | |
| Diphenylanthracene | 375 | 400 |
| 1,4-bis(5-phenyl-2 oxazolyl)benzene (POPOP) | 359 | 380-440 |

Examples of solvents that may be used in the fabrication of the scintillant-doped nanoparticles include, but are not limited to, tetrahydrofuran, isopropanol, chloroform, acetonitrile, ethyl acetate, toluene, acetone, butanol, ethanol, dichloromethane, or a combination thereof.

In some embodiments, the scintillant fluorophores may be incorporated into the polymer matrix at ratios that range from about 1:1 to 1:10, 1:10 to 1:100, 1:1 to 10:1, or 10:1 to 100:1, e.g. ratio of scintillant fluorophore 1 to scintillant fluorophore 2 is about 20:1. In some embodiments, an amount of organic solvent to aqueous dispersion of nanoparticles ranges from about 1% to 50% (v/v). In some embodiments, the aqueous dispersion of nanoparticles may contain about 1 mg/mL to 100 mg/mL polymer core nanoparticles. It is understood that these values are non-limiting and that other ratios may also be used.

Without wishing to limit the present invention to any theory or mechanism, the scintillant nanoparticles may detect radioactivity when energy is transferred from the polymer to scintillant fluorophore 1 or scintillant fluorophore 2, and energy from scintillant fluorophore 1 is transferred to scintillant fluorophore 2.

### Example 8

The following are examples of producing scintillant-doped polystyrene core nanoparticles using the swelling-deswelling process with varying solvent volumes. Equivalents or substitutes are within the scope of the present invention.

Lower solvent volume: Scintillant fluorophores pTP (40 mM) and dimethyl POPOP (4 mM) were dissolved in 6 mL chloroform:isopropanol (9:1, v:v) Scintillant fluorophores dissolved in solvent were added directly to the aqueous polystyrene nanoparticle solution (60 mg/mL, 100 mL total volume) in a 500 mL round-bottomed flask. The nanoparticle solution was sonicated using a bath sonicator for several minutes to disperse solvent droplets throughout the water. A small amount of solvent was then removed under reduced pressure using a rotary evaporator. The nanoparticles solution was sonicated again for several minutes, followed by further solvent removal under reduced pressure. This sonication/solvent removal process was repeated for a total of 5 cycles, until as much organic solvent was removed as possible.

Higher Solvent Volume: Scintillant fluorophores pTP (40 mM) and dimethyl POPOP (4 mM) were dissolved in 20 mL chloroform:isopropanol (9:1, v:v) Scintillant fluorophores dissolved in solvent were added directly to the aqueous polystyrene nanoparticle solution (60 mg/mL, 100 mL total volume) in a 500 mL round-bottomed flask. The nanoparticle solution was sonicated using a bath sonicator for several minutes to disperse solvent droplets throughout the water. A small amount of solvent was then removed under reduced pressure using a rotary evaporator. The nanoparticles solution was sonicated again for several minutes, followed by further solvent removal under reduced pressure. This sonication/solvent removal process was repeated for a total of 5 cycles, until as much organic solvent was removed as possible. The swelling/deswelling method has a total polymer yield of about 30-50%.

### Example 9

The following are further examples of producing scintillant-doped polystyrene core nanoparticles using the swelling-deswelling process. It is notable that some scintillators do not incorporate into the polymer nanoparticles using the inclusion method. However, it was surprisingly found that these scintillators did incorporate into the polymer nanoparticles using the swelling-deswelling method. Equivalents or substitutes are within the scope of the present invention.

Fabrication of Decacyclene-Doped Polystyrene Core nPs

Decacyclene does not incorporate into the polymer using the inclusion method described above, thus the swelling/deswelling method had to be used. The scintillant fluorophores pTP (2.9 mM) and decacyclene (27 μM) were dissolved in 3 mL chloroform:isopropanol (9:1, v:v). Scintillant fluorophores dissolved in solvent (1 mL) were added directly to the aqueous polystyrene nanoparticle solution (approximately 15 mg/mL, 8 mL total volume) in a 500 mL round-bottomed flask. The nanoparticle solution was sonicated using a bath sonicator for several minutes to disperse solvent droplets throughout the water. A small amount of solvent was then removed under reduced pressure using a rotary evaporator. The remaining 2 mL of scintillant fluorophores dissolved in solvent was added to the nanoparticle solution. The nanoparticles solution was sonicated again for several minutes, followed by further solvent removal under reduced pressure.

### Fabrication of tris(1,3-diphenyl-1,3-propanedionato)-(1,10-phenanthroline) europium (I11)-Doped Polystyrene Core nPs

The swelling/deswelling method was used for the scintillant fluorophore tris(1,3-diphenyl-1,3-propanedionato)-(1,10-phenanthroline) europium (Ill) because it was more expensive than other scintillant fluorophores and the swelling/deswelling process could be controlled better than the inclusion method.

The scintillant fluorophores PMP (0.38 M) and tris(1,3-diphenyl-1,3-propanedionato)-(1,10-phenanthroline) europium (Ill) (6 mM) were dissolved in 1 mL THF. Scintillant fluorophores dissolved in solvent were added directly to the aqueous polystyrene nanoparticle solution (approximately 60 mg/mL, 10 mL total volume) in a 500 mL round-bottomed flask. The nanoparticle solution was sonicated using a bath sonicator for several minutes to disperse solvent droplets throughout the water. A small amount of solvent was then removed under reduced pressure using a rotary evaporator. The nanoparticles solution was sonicated again for several minutes, followed by further solvent removal under reduced pressure. This sonication/solvent removal process was repeated for a total of 5 cycles, until as much organic solvent was removed as possible.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A method for producing scintillant-doped polymer core nanoparticles for detecting radioisotope activity, the method comprising:
a. polymerizing monomers to form polymer nanoparticles; and
b. doping the polymer nanoparticles with one or more scintillators to form the scintillant-doped polymer core nanoparticles.

2. The method of claim 1, wherein one or more scintillators are benzene, naphthalene, anthracene, tetracene, substituted benzenes, substituted naphthalenes, substituted anthracenes, substituted tetracenes, substituted pyrazolines, substituted oxazoles, substituted phenyloxazolyls, substituted quinolines, or a combination thereof.

3. The method of claim 1 further comprising mixing silica precursors with the scintillant-doped polymer core nanoparticles to form a functionalized silica shell that encapsulates each scintillant-doped polymer core nanoparticle, thereby forming scintillation nanoparticles.

4. The method of claim 3 further comprising depositing a lipid bilayer on an outer surface of the scintillation nanoparticle such that the outer surface is substantially covered by the lipid bilayer.

5. The method of claim 4 further comprising embedding receptors in the lipid bilayer.

6. The method of claim 5, wherein the receptors are membrane protein receptors, growth factor receptors, G-protein coupled receptors, ion channels, lipid-derived receptors, glycoprotein receptors, glycolipids, phospholipids, or a combination thereof.

7. A method for detecting radioisotope activity in a sample, the method comprising:
a. preparing scintillant-doped polymer core nanoparticles according to claim 1;
b. combining the scintillant-doped polymer core nanoparticles and the sample in a medium, wherein radioactive decay of the radioisotopes in the sample generate energetic particles that interact with the scintillant-doped nanoparticles, resulting in the emission of photons; and
c. counting the photon emissions.

8. The method of claim 7, wherein the energetic particles are β-particles.

9. The method of claim 7, wherein the medium is an aqueous solution.

10. A method for producing scintillant-doped polymer core nanoparticles for detecting radioisotope activity, the method comprising:
a. adding monomers to an aqueous solution;
b. polymerizing the monomers to form polymer core nanoparticles in solution;
c. dissolving scintillators in an organic solvent;
d. adding the scintillators in the organic solvent to the polymer core nanoparticle solution;
e. agitating the mixture of the scintillators in the organic solvent and the polymer core nanoparticle solution, thereby doping the polymer core nanoparticles with the scintillators to form scintillant-doped polymer nanoparticles; and
f. removing the organic solvent from the mixture, thereby forming a concentrated solution of scintillant-doped polymer nanoparticles.

11. The method of claim 10, wherein removing the organic solvent from the mixture comprises:
a. evaporating a portion of the organic solvent;
b. agitating the remaining mixture; and
c. repeating steps a. and b. for a number of iterations to allow for improved loading by increasing the contact of the scintillators with the polymer core nanoparticles as the organic solvent is gradually removed.

12. The method of claim 10 further comprising:
a. redispersing the concentrated solution of scintillant-doped polymer nanoparticles in a second solvent having a base; and
b. mixing silica precursors into the scintillant-doped polymer nanoparticles dispersed in the second solvent, wherein the silica precursors form a functionalized silica shell that encapsulates each scintillant-doped polymer nanoparticle, thereby forming scintillation nanoparticles.

13. The method of claim 12, wherein the base is effective for tuning the thickness of the silica shell, wherein the base has a pH ranging from 8 to 12.

14. The method of claim 12 further comprising depositing a lipid bilayer on an outer surface of the scintillation nanoparticle such that the outer surface is substantially covered by the lipid bilayer.

15. The method of claim 14 further comprising embedding receptors in the lipid bilayer.

16. The method of claim 15, wherein the receptors are membrane protein receptors, growth factor receptors, G-protein coupled receptors, ion channels, lipid-derived receptors, glycoprotein receptors, glycolipids, phospholipids, or a combination thereof.

17. The method of claim 10, wherein the scintillators are benzene, naphthalene, anthracene, tetracene, substituted benzenes, substituted naphthalenes, substituted anthracenes, substituted tetracenes, substituted pyrazolines, substituted oxazoles, substituted phenyloxazolyls, substituted quinolines, or a combination thereof.
